(19) United States
(12) Patent Application Publication
(10) Pub. No.: US 2006/0199857 A1
(43) Pub. Date: Sep. 7, 2006

(54) Therapeutic agents

(75) Inventors: Eva-Lotte Lindstedt-Alstermark, Molndal (SE); Anna Maria Persdotter Boije, Molndal (SE); Patrik Holm, Molndal (SE)

Correspondence Address: MORGAN LEWIS & BOCKIUS LLP, 1111 PENNSYLVANIA AVENUE NW, WASHINGTON, DC, 20004, US

(73) Assignee: ASTRAZENECA UK LIMITED, London (GB)

(21) Appl. No.: 10/561,126

(22) PCT Filed: Jun. 16, 2004
(86) PCT No.: PCT/GB04/02554

§ 371 Date: Dec. 16, 2005

(30) Foreign Application Priority Data
Jun. 18, 2003 (GB) 0314075.3

(51) Int. Cl. A61K 31/381; A61K 31/277; A61K 31/192; C07C 317/30; A61K 31/195

(52) U.S. Cl. 514/445; 558/410; 562/429; 549/62; 514/520; 514/568; 514/562

(57) ABSTRACT

Substituted 3-phenylpropionic acid derivatives, processes for preparing such compounds, their utility in treating clinical conditions including lipid disorders (dyslipidemias) whether or not associated with insulin resistance, methods for their therapeutic use and pharmaceutical compositions containing them.

### FIELD OF THE INVENTION

The present invention relates to certain novel substituted 3-phenylpropionic acid derivatives, to processes for preparing such compounds, to their utility in treating clinical conditions including lipid disorders (dyslipidemias) whether or not associated with insulin resistance and other manifestations of the metabolic syndrome, to methods for their therapeutic use and to pharmaceutical compositions containing them

### BACKGROUND OF THE INVENTION

The metabolic syndrome including type 2 diabetes mellitus, refers to a cluster of manifestations including insulin resistance with accompanying hyperinsulinaemia, possibly type 2 diabetes mellitus, arterial hypertension, central (visceral) obesity, dyslipidaemia observed as deranged lipoprotein levels typically characterised by elevated VLDL (very low density lipoproteins), small dense LDL particles and reduced HDL (high density lipoprotein) concentrations and reduced fibrinolysis.

Recent epidemiological research has documented that individuals with insulin resistance run a greatly increased risk of cardiovascular morbidity and mortality, notably suffering from myocardial infarction and stroke. In type 2 diabetes mellitus atherosclerosis related conditions cause up to 80% of all deaths.

In clinical medicine there is awareness of the need to increase the insulin sensitivity in patients with the metabolic syndrome and thus to correct the dyslipidaemia which is considered to cause the accelerated progress of atherosclerosis. However, currently this is not a universally accepted diagnosis with well-defined pharmacotherapeutic indications.

2-Phenylpropionic acids and cinnamic acids derivatives, unsubstituted in their acid chains, are disclosed in WO95/15752 and EP 544 488 as leukotriene antagonists and EP 947 500 discloses similar compounds which also have a sulphonamide or carboxamide group have prostaglandin E₂ modulating activity.

The S-enantiomer of the compound of formula C below 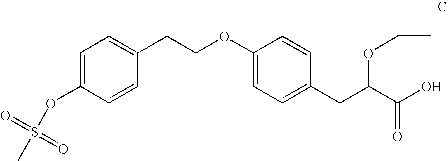
2-ethoxy-3-[4-(2-{4-methanesulfonyloxyphenyl}ethoxy)phenyl]propanoic acid, is disclosed in PCT Publication Number WO99/62872. This compound is reported to be a modulator of peroxisome proliferator-activated receptors (PPAR, for a review of the PPARs see T. M. Willson et al, J Med Chem 2000, Vol 43, 527) and has combined PPARα/PPARγ agonist activity (Structure, 2001, Vol 9, 699, P. Cronet et al). This compound is effective in treating conditions associated with insulin resistance. Other 2-phenylpropionic acid derivatives are disclosed in WO99/62870, WO99/62871 and WO01/40172. 2-Phenylpropanol derivatives having PPAR activity are disclosed in WO01/40170 and WO02/96863.

2-Chloro-2-((4-phenoxyalkyl)phenyl)propionic acids derivatives are disclosed as having hypolipidemic and hypoglycaemic properties in GB 1,496,156.

(S)-2-Ethoxy-3-[4-(4-methylsulfonyloxyphenethylamino)phenyl]propionic acid is disclosed WO03/048116 which describes compounds that are predominantly PPAR alpha agonists.

2-Alkoxy-3-[(4-(2-quinolinylmethoxy)phenoxyalkylphenyl]propionic acid derivatives are described as having PPAR activity in WO01/66098.

WO00/64888 discloses diaryl acid derivatives as PPAR receptor ligands.

WO02/100813 discloses 2-alkoxy-3-{4-[(4-substitutedphenylphenoxy)-alkyl]phenyl}propionic acid compounds that have PPAR activity.

EP 1 216 980 discloses 2-alkoxy-3-{3-[(4-substitutedphenoxy)alkyl]phenyl}propionic acid compounds that have PPAR activity.

Co-pending PCT application No. PCT/GB02/05743 discloses compounds of formula I 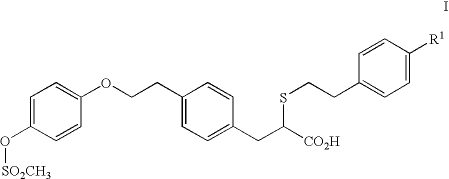
wherein R¹ represents chloro, fluoro or hydroxy as well as optical isomers and racemates thereof as well as pharmaceutically acceptable salts, prodrugs, solvates and crystalline forms thereof, to processes for preparing such compounds, to their the utility in treating clinical conditions including lipid disorders (dyslipidemias) whether or not associated with insulin resistance, to methods for their therapeutic use and to pharmaceutical compositions containing them.

Surprisingly a series of compounds has now been found which are PPARα and/or PPARγ modulators.

### DESCRIPTION OF THE INVENTION

The present invention provides a compound of formula I 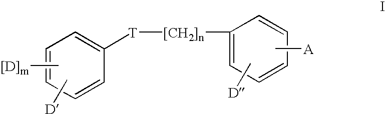
and pharmaceutically acceptable salts thereof, in which
A is situated in the para position and represents A1 or A2 below 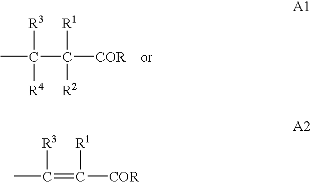
wherein
R is hydrogen;
—OR^{a}, wherein R^{a} represents hydrogen, alkyl, aryl or alkylaryl;
—NR^{a}R^{b}, wherein R^{a} and R^{b} are the same or different and R^{a} is as defined above and R^{b} represents hydrogen, alkyl, aryl, alkylaryl, cyano, —OH, -Oalkyl, -Oaryl, -Oalkylaryl, —COR^{c} or —SO₂R^{d}, wherein R^{c} represents hydrogen, alkyl, aryl or alkylaryl and R^{d} represents alkyl, aryl or alkylaryl;
   R¹ is alkyl, aryl, alkenyl, alkynyl, cyano;
—OR^{e}, wherein R^{e} is alkyl acyl, aryl or alkylaryl;
—O—[CH₂]ₘ—OR^{f}, wherein R^{f} represents hydrogen, alkyl, acyl, aryl or alkylaryl and m represents an integer 1-8;
—OCONR^{a}R^{c}, wherein R^{a} and R^{c} are as defined above;
—SR^{d}, wherein R^{d} is as defined above;
—SOR^{d}, wherein R^{d} is as defined above;
—SO₂R^{d}, wherein R^{d} is as defined above;

—SO₂NR^{a}R^{f}, wherein R^{f} and R^{a} are as defined above;
—SO₂OR^{a}, wherein R^{a} is as defined above;
—COOR^{d}, wherein R^{d} is as defined above;
   R is hydrogen, alkyl, aryl, or alkylaryl,
   R³ and R⁴ are the same or different and each represents hydrogen, alkyl, aryl, or alkylaryl,
   n is an integer 1-6,
   m is an integer 0 or 1;
   D is situated in the ortho, meta or para position and represents alkyl, acyl, aryl, alkylaryl, halogen, —CN and NO₂, wherein the alkyl, aryl, or alkylaryl group is optionally substituted by R^{b};
   —NR^{c}COOR^{a}, wherein R^{c} and R^{a} are as defined above;
   —NR^{c}COR^{a}, wherein R^{c} and R^{a} are as defined above;
   —NR^{c}R^{a}, wherein R^{c} and R^{a} are as defined above;
   —NR^{c}SO₂R^{d}, wherein R^{c} and R^{d} are as defined above;
   —NR^{c}CONR^{k}R^{c}, wherein R^{a}, R^{c} and R^{k} are as defined above;
   —NR^{c}CSNR^{a}R^{k}, wherein R^{a}, R^{c} and R^{k} are as defined above;
   —OR^{a}, wherein R^{a} is as defined above;
   —OSO₂R^{d}, wherein R^{d} is as defined above;
   —SO₂R^{d}, wherein R^{d} is as defined above;
   —SOR^{d}, wherein R^{d} is as defined above;
   —SR^{c}, wherein R^{c} is as defined above;
   —SO₂NR^{a}R^{f}, wherein R^{f} and R^{a} are as defined above;
   —SO₂OR^{a}, wherein R^{a} is as defined above;
   —CONR^{c}R^{a}, wherein R^{c} and R^{a} are as defined above;
   —OCONR^{f}R^{a}, wherein R^{f} and R^{a} are as defined above;
   D′ is situated in the ortho, meta or para position and represents hydrogen, alkyl, acyl, aryl, alkylaryl halogen, —CN, —NO₂,
   —NR^{f}R^{b}, wherein R^{f} and R^{b} are as defined above;
   —OR^{f}, wherein R^{f} is as defined above;
   —OSO₂R^{d}, wherein R^{d} is as defined above;
   D″ is situated in the ortho, meta or para position and represents
hydrogen, alkyl acyl, aryl, alkylaryl, halogen, —CN, —NO₂, —NR^{f}R^{b} wherein R^{f} and R^{b} are as defined above;
—OR^{f}, wherein R^{f} is as defined above.
—OSO₂R^{d}, wherein R^{d} is as defined above
   and T represents O, S or NR^{t} wherein R^{t} represents alkyl or alkylaryl provided that when A is A1 and R², R³, and R⁴ each represent hydrogen and R¹ is OR^{e} wherein R^{e} is as previously defined then T is not O;
   wherein the term “aryl” denotes a substituted or unsubstituted phenyl, furyl, thienyl or pyridyl group, or a fused ring system of any of these groups;
   wherein the term “alkyl” denotes a straight or branched, substituted or unsubstituted alkyl group having from 1 to 6 carbon atoms or a substituted or unsubstituted cycloalkyl having from 3 to 6 carbon atoms and wherein the term “substituted” denotes substitution by one or more alkyl, alkoxy, halogen, thiol, nitro, hydroxy, acyl, aryl or cyano groups or an amino group optionally substituted by one or two alkyl groups:

with a first proviso that when D is CH₃S(O)₂O and m is 1 and D′ is H and T is O and n=2 and A is a group CH₂CH(SCH₂CH₂Ph)COR^{x} in which the phenyl is substituted in the 4 position by OH, Cl or F and in which R^{x} represents OH, or a protecting group for a carboxylic hydroxy group including a ethoxy or benzyloxy then D″ is not H;

and with a second proviso that when m is 1 and D is CH₃S(O)₂O and D′ is H and T is O, S or NR and wherein R represents a H, a C₁₋₆alkyl group or a phenyl C₁₋₆alkyl group and n=2 and A is a group CH₂CH(OC₂H₅)COR^{x} in which R^{x} represents OH, or a protecting group for a carboxylic hydroxy group including a C₁₋₆alkoxy group or benzyloxy then D″ is not H.

The present invention provides a compound of formula I 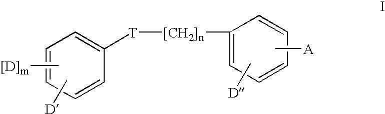
and pharmaceutically acceptable salts thereof, in which
A is situated in the ortho, meta or para position and represents 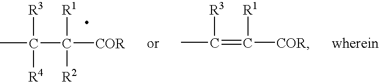
   R is hydrogen;
—OR^{a}, wherein R^{a} represents hydrogen, alkyl, aryl or alkylaryl;
—NR^{a}R^{b}, wherein R^{a} and R^{b} are the same or different and R^{a} is as defined above and R^{b} represents hydrogen, alkyl, aryl, alkylaryl, cyano, —OH, -Oalkyl, -Oaryl, -Oalkylaryl, —COR^{c} or —SO₂R^{d}, wherein R^{c} represents hydrogen, alkyl, aryl or alkylaryl and R^{d} represents alkyl, aryl or alkylaryl;
   R¹ is alkyl aryl, alkenyl, alkynyl, cyano;
—OR^{e}, wherein R^{e} is alkyl, acyl, aryl or alkylaryl;
—O—[CH₂]ₘ—OR^{f}, wherein R^{f} represents hydrogen, alkyl, acyl, aryl or alkylaryl and m represents an integer 1-8;
—OCONR^{a}R^{c}, wherein R^{a} and R^{c} are as defined above;
—SR^{d}, wherein R^{d} is as defined above;
—SO₂NR^{a}R^{f}, wherein R^{f} and R^{a} are as defined above;
—SO₂OR^{a}, wherein R^{a} is as defined above;
—COOR^{d}, wherein R^{d} is as defined above;
   R² is hydrogen, halogen, alkyl, aryl, or alkylaryl,
   R³ and R⁴ are the same or different and each represents hydrogen, alkyl, aryl, or alkylaryl,
   n is an integer 1-6,
   m is an integer 0 or 1 (preferably m is 1);
   D is situated in the ortho, meta or para position (preferably D is situated in the para position) and represents alkyl, acyl, aryl, alkylaryl, halogen, —CN and NO₂, wherein the alkyl, aryl, or alkylaryl group is optionally substituted by R^{b};
   —NR^{c}COOR^{a}, wherein R^{c} and R^{a} are as defined above;
   —NR^{c}COR^{a}, wherein R^{c} and R^{a} are as defined above;
   —NR^{c}R^{a}, wherein R^{c} and R^{a} are as defined above;
   —NR^{c}SO₂R^{d}, wherein R^{c} and R^{d} are as defined above;
   —NR^{c}CONR^{k}R^{c}, wherein R^{a}, R^{c} and R^{k} are as defined above;
   —NR^{c}CSNR^{a}R^{k}, wherein R^{a}, R^{c} and R^{k} are as defined above;
   —OR^{a}, wherein R^{a} is as defined above;
   —OSO₂R^{d}, wherein R^{d} is as defined above;
   —SO²R^{d}, wherein R^{d} is as defined above;
   —SOR^{d}, wherein R^{d} is as defined above;
   —SR^{c}, wherein R^{c} is as defined above;
   —SO₂NR^{a}R^{f}, wherein R^{f} and R^{a} are as defined above;
   —SO₂OR^{a}, wherein R^{a} is as defined above;
   —CONR^{c}R^{a}, wherein R^{c} and R^{a} are as defined above;
   —OCONR^{f}R^{a}, wherein R^{f} and R^{a} are as defined above;
   D′ is situated in the ortho, meta or para position (preferably D′ is situated in the ortho or meta position) and represents hydrogen, alkyl, acyl, aryl, alkylaryl, halogen, —CN, —NO₂,
   —NR^{f}R^{b}, wherein R^{f} and R^{b} are as defined above;
   —OR^{f}, wherein R^{f} is as defined above;
   —OS₂R^{d}, wherein R^{d} is as defined above;
   D″ is situated in the ortho, meta or para position and represents
hydrogen, alkyl, acyl, aryl, alkylaryl, halogen, —CN, —NO₂, —NR^{f}R^{b} wherein R^{f} and R^{b} are as defined above;
—OR^{f}, wherein R^{f} is as defined above.
—OSO₂R^{d}, wherein R^{d} is as defined above
   and T represents O, S or NR^{t} wherein R^{t} represents alkyl or alkylaryl;
   wherein the term “aryl” denotes a substituted or unsubstituted phenyl, furyl, thienyl or pyridyl group, or a fused ring system of any of these groups;
   wherein the term “alkyl” denotes a straight or branched, substituted or unsubstituted alkyl group having from 1 to 6 carbon atoms or a substituted or unsubstituted cycloalkyl having from 3 to 6 carbon atoms and wherein the term “substituted” denotes substitution by one or more alkyl, alkoxy, halogen, thiol, nitro, hydroxy, acyl, aryl or cyano groups or an amino group optionally substituted by one or two alkyl groups:

with a first proviso that when D is CH₃S(O)₂O and D′ is H and T is O and n=2 and A is a group CH₂CH(SCH₂CH₂Ph)COR^{x} in which the phenyl is substituted in the 4 position by OH, Cl or P and in which R^{x} represents OH, or a protecting group for a carboxylic hydroxy group including a ethoxy or benzyloxy then D″ is not H; and

a second proviso that when m is 1 and D is CH₃S(O)₂O and D′ is H and T is O, S or NR and wherein R represents a H, a C₁₋₆alkyl group or a phenyl C₁₋₆alkyl group and n=2 and A is a group CH₂CH(OC₂H₅)COR^{x} in which R^{x} represents OH, or a protecting group for a carboxylic hydroxy group including a C₁₋₆alkoxy group or benzyloxy then D″ is not H.

Preferably m is 1.

Preferably D is situated in the para position.

Preferably D′ is situated in the ortho or meta position.

Further values of T, D and A in compounds of Formula I now follow. It will be understood that such values may be used where appropriate with any of the definitions, claims or embodiments defined hereinbefore or hereinafter.

In a first group of compounds of formula I, T is O.

In a second group of compounds of formula I, T is S.

In a third group of compounds of formula I T is NH.

In a fourth group of compounds of formula I, A is a group CH₂CH(R^{y})CO₂H in which R^{y} represents arylethylthio in which the aryl is optionally substituted by one or more of the following, C₁₋₆alkyl, C₁₋₆alkoxy, halo, cyano or an amino group optionally substituted by one or two alkyl groups.

In a fifth group of compounds of formula I, m is 1 and D is methanesulphonyloxy.

In a sixth group of compounds of formula I, A represents a group of formula CH₂—CH(CO₂H)—S(O)ₚ—(CH₂)_{q}—Ar wherein p is 0, 1 or 2; q is 1, 2, 3 or 4; and

Ar is phenyl or thienyl each of which is optionally substituted by one or more hydroxy, C₁₋₆alkyl, C₁₋₆alkoxy, halogen, cyano or an amino group optionally substituted by one or two alkyl groups.

In another aspect the present invention provides a compound of formula IA 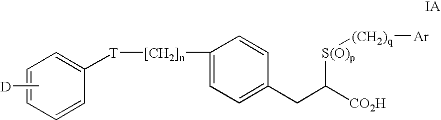
or a pharmaceutically acceptable salt thereof in which
D represents C₁₋₆alkylsulfonyloxy, aroyl, benzyl or a C₁₋₆alkyl group;
T represents O, S or NR^{t} wherein R^{t} represents alkyl or alkylaryl;
n is 1, 2 or 3;
p is 0, 1 or 2;
q is 1 or 2; and
Ar is phenyl or thienyl each of which is optionally substituted by hydroxy, C₁₋₆alkyl, C₁₋₆alkoxy, halo, cyano or an amino group optionally substituted by one or two alkyl groups and wherein the group containing the carboxylic acid group is attached to the phenyl ring meta or para to the group (CH₂)n-T-.

In a particular group of compounds of formula IA n is 2.

In a particular group of compounds of formula IA T is O.

In a particular group of compounds of formula IA D is CH₃SO₂O, particularly in the para position to T

It will be appreciated by those skilled in the art the compounds of formula I contain an optically active centre and therefore can exist as enantiomers which can be separated as described later. It is expected that most, if not all, of the activity of the compounds of formula I resides in one enantiomer: either the S or the R enantiomer or the (+) or the (−) enantiomer. The enantiomers which are more active in the assays which are described later are preferred forms of the present invention. It will be understood that the present invention includes all mixtures of this active enantiomer with the other enantiomer, for example the racemic mixture, which is a useful intermediate for the active enantiomer.

The active enantiomers may be isolated by separation of racemate for example by fractional crystallization, resolution or HPLC on a chiral column (for example a Chiralpak™ AD 250×50 column). Alternatively the active enantiomers may be made by chiral synthesis from chiral starting materials under conditions which will not cause racemisation or epimerisation, or by derivatisation with a chiral reagent.

The following definitions shall apply throughout the specification and the appended claims with regard to the group A.

Unless otherwise stated or indicated, the term “alkyl” denotes a straight or branched, substituted or unsubstituted alkyl group having from 1 to 6 carbon atoms or a cyclic alkyl having from 3 to 6 carbon atoms. The term “lower alkyl” denotes a straight or branched, substituted or unsubstituted alkyl group having from 1 to 3 carbon atoms or a cyclic alkyl having 3 carbon atoms. Examples of said alkyl and lower alkyl include methyl, ethyl, n-propyl, isopropyl, n-butyl, iso-butyl, sec-butyl, t-butyl and straight- and branched-chain pentyl and hexyl as well as cyclopropyl cyclobutyl, cyclopentyl and cyclohexyl.

Unless otherwise stated or indicated, the term “alkoxy” denotes a group O-alkyl, wherein alkyl is as defined above.

Unless otherwise stated or indicated, the term “halogen” shall mean fluorine, chlorine, bromine or iodine.

Unless otherwise stated or indicated, the term “aryl” denotes a substituted or unsubstituted phenyl, furyl, thienyl or pyridyl group, or a fused ring system of any of these groups, such as naphthyl. Preferably aryl is a substituted or unsubstituted phenyl.

Unless otherwise stated or indicated, the term “substituted” denotes an alkyl or an aryl group as defined above which is substituted by one or more alkyl, alkoxy, halogen, amino, thiol, nitro, hydroxy, acyl, aryl or cyano groups.

Unless otherwise stated or indicated, the term “alkylaryl” denotes a 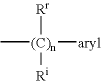
wherein n is an integer 1 to 6 and R^{r} and Rⁱ are the same or different and each represents hydrogen or an alkyl or aryl group as defined above.

Unless otherwise stated or indicated, the term “acyl” denotes a group 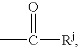
wherein R^{j} is hydrogen, alkyl, aryl and alkylaryl as defined above.

Unless otherwise stated or indicated, the terms “alkenyl” and “alkynyl” denote a straight or branched, substituted or unsubstituted unsaturated hydrocarbon group having one or more double or triple bonds and having a maximum of 6 carbon atoms, preferably 3 carbon atoms.

Unless otherwise stated or indicated the term “protective group” (R^{p}) denotes a protecting group as described in the standard text “Protecting groups in Organic Synthesis”, 2nd Edition (1991) by Greene and Wuts. The protective group may also be a polymer resin such as Wang resin or 2-chlorotrityl chloride resin.

“Aroyl” means phenyl-(CO)—.

The term “prodrug” as used in this specification includes derivatives of the carboxylic acid group which are converted in a mammal, particularly a human, into the carboxylic acid group or a salt or conjugate thereof. The term “prodrug” also includes derivatives of the hydroxy substituent (when R¹ represents hydroxy) which are converted in a mammal, particularly a human, into the hydroxy group or a salt or conjugate thereof. It should be understood that, whilst not being bound by theory, it is believed that most of the activity associated with the prodrugs arises from the activity of the compound of formula I into which the prodrugs are converted. Prodrugs can be prepared by routine methodology well within the capabilities of someone skilled in the art. Various prodrugs of carboxy and hydroxy are known in the art. For examples of such prodrug derivatives, see:
a) Design of Prodrugs, edited by H. Bundgaard, (Elsevier, 1985) and Methods in Enzymology. 42: 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8:1-38 (1992);
d) H. Bundgaard, et al., Journal of Pharmaceutical Sciences, 77:285 (1988); and
e) N. Kakeya, et al., Chem Pharm Bull, 32:692 (1984).

The above documents a to e are herein incorporated by reference.

In vivo cleavable esters are just one type of prodrug of the parent molecule. An in vivo hydrolysable (or cleavable) ester of a compound of the formula (I) that contains a carboxy or a hydroxy group is, for example, a pharmaceutically acceptable ester which is hydrolysed in the human or animal body to produce the parent acid or alcohol. Suitable pharmaceutically acceptable esters for carboxy include C₁₋₆alkoxymethyl esters, for example, methoxymethyl; C₁₋₆alkanoyloxymethyl esters, for example, pivaloyloxymethyl; phthalidyl esters; C₃₋₈cycloalkoxycarbonyloxyC₁₋₆alkyl esters, for example, 1-cyclohexyl-carbonyloxyethyl; 1,3-dioxolen-2-onylmethyl esters, for example, 5-methyl-1,3-dioxolen-2-onlymethyl; and C₁₋₆alkoxycarbonyloxyethyl esters, for example, 1-methoxycarbonyloxyethyl; and may be formed at any carboxy group in the compounds of this invention. An in vivo hydrolysable (or cleavable) ester of a compound of the formula (I) that contains a hydroxy group includes inorganic esters such as phosphate esters (including phosphoramidic cyclic esters) and α-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

The compounds of formula I have activity as medicaments, in particular the compounds of formula I are agonists of PPARα and PPARγ.

Specific compounds of the invention are one or more of the following:
2-[(4-cyanobenzyl)thio]-3-[4-(2-{4[(methylsulfonyl)oxy]phenoxy}ethyl)-phenyl]propanoic acid;
2-({2-[4-(dimethylamino)phenyl]ethyl}thio)-3-[4-(2-{4-[(methylsulfonyl)oxy]phenoxy}-ethyl)phenyl]propanoic acid;
3-[4-(2-{4-[(methylsulfonyl)oxy]phenoxy}ethyl)phenyl]-2-{[2-(2-thienyl)ethyl]thio}-propanoic acid;
2-{[2-(2-fluorophenyl)ethyl]thio}-3-[4-(2-{4-[(methylsulfonyl)oxy]phenoxy}-ethyl)phenyl]-propanoic acid;
2-{[2-(3-methoxyphenyl)ethyl]thio}-3-[4-(2-{4-[(methylsulfonyl)oxy]phenoxy}-ethyl)phenyl]propanoic acid;
2-{[2-(4-hydroxyphenyl)ethyl]sulfinyl}-3-[4-(2-{4-[(methylsulfonyl)oxy]phenoxy}-ethyl)phenyl]propanoic acid;
3-{4-[2-(4-benzoylphenoxy)ethyl]phenyl}-2-{[2-(4-hydroxyphenyl)ethyl]thio}propanoic acid;
methyl 2-({2-[4-(benzyloxy)phenyl]ethyl}thio)-3-{4-[2-(2-propylphenoxy)ethyl]phenyl}-propanoate;
2-{[2-(4-hydroxyphenyl)ethyl]thio}-3-{4-[2-(2-propylphenoxy)ethyl]phenyl}-propanoic acid;
2-{[2-(4-hydroxyphenyl)ethyl]thio}-3-[3-(2-{4-[(methylsulfonyl)oxy]phenoxy}-ethyl)phenyl]propanoic acid;
3-{4-[2-(2-benzyl-4-methanesulfonyloxyphenoxy)ethyl]phenyl}-2-[2-(4-hydroxyphenyl)-ethylsulfanyl]propionic acid; and
2-[2-(4-tert-butoxy-phenyl)ethylsulfanyl]-3-{4-[2-(4-methanesulfonyloxyphenoxy)ethyl]-phenyl}propionic acid
   and pharmaceutically acceptable salts thereof.

It will also be understood that certain compounds of the present invention may exist in solvated, for example hydrated, as well as unsolvated forms. It is to be understood that the present invention encompasses all such solvated forms. Certain compounds of the present invention may exist as tautomers. It is to be understood that the present invention encompasses all such tautomers.

Methods of Preparation

The compounds of the invention may be prepared as described in the Examples and analogous methods thereto known to persons skilled in the art. In particular methods disclosed in WO 99/62871 and analogous methods thereto may be used. However, the invention is not limited to these methods, the compounds may also be prepared as described for structurally related compounds in the prior art. The reactions can be carried out according to standard procedures or as described in the experimental section.

Compounds of formula I may be prepared by reacting a compound of formula II 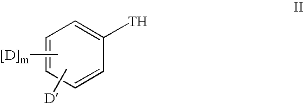
in which D, m, D′ and T are as previously defined with a compound of formula III 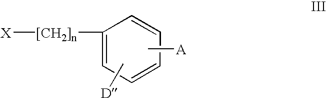
in which n, A and D″ are as previously defined and X is a leaving group for example halo or methanesulphonyloxy at a temperature in the range of 0-150° C. optionally on the presence of an inert sovent. Optionally protection and deprotection steps known to those skilled in the art may be used as as necessary.

Compounds of formula I in which T is O may be prepared by reacting a compound of formula IV 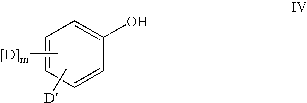
in which D, m and D′ are as previously defined with a compound of formula V 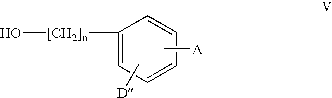
in which n, A and D″ are as previously defined using Mitsonobu conditions known to those skilled in the art for example in the presence of a coupling agent, for example cyanomethylenetri-N-butylphosphorane.

Compounds of formula IA may be prepared by reacting a compound of formula IB 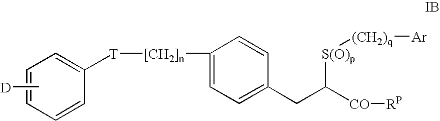
in which D, T, n, p, q and Ar are as previously defined and R^{p} represents a protecting group for a carboxylic hydroxy group as described in the standard text “Protective Groups in Organic Synthesis”, 3rd Edition (1999) by Greene and Wuts, with a de-protecting agent. The protecting group may also be a resin, such as Wang resin or 2-chlorotrityl chloride resin. Protecting groups may be removed in accordance to techniques which are well known to those skilled in the art. One such protecting group is where R^{p} represents a C₁₋₆alkoxy group for example methoxy or ethoxy or an arylalkoxy group eg benzyloxy, such that COR⁴ represents an ester. Such esters can be reacted with a de-protecting agent e.g. a hydrolysing agent, for example lithium hydroxide in a mixture of THF and water, at a temperature in the range of 0-100° C. to give compounds of formula I.

Compounds of formula II, III, IV, and V may be prepared by methods known to those skilled in the art see for example WO 99/62871 herein incorporated by reference.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

The expression “inert solvent” refers to a solvent that does not react with the starting materials, reagents, intermediates or products in a manner which adversely affects the yield of the desired product.

Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient either as a free acid, or a pharmaceutical acceptable organic or inorganic base addition salt, in a pharmaceutically acceptable dosage form. Depending upon the disorder and patient to be treated and the route of administration, the compositions may be administered at varying doses.

Suitable daily doses of the compounds of the invention in therapeutical treatment of humans are about 0.0001-100 mg/kg body weight, preferably 0.001-10 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is thus provided a pharmaceutical formulation including any of the compounds of the invention, or pharmaceutically acceptable derivatives thereof, in admixture with pharmaceutically acceptable adjuvants, diluents and/or carriers.

Pharmacological Properties

The present compounds of formula (I) are useful for the prophylaxis and/or treatment of clinical conditions associated with inherent or induced reduced sensitivity to insulin (insulin resistance) and associated metabolic disorders (also known as metabolic syndrome). These clinical conditions win include, but win not be limited to, general obesity, abdominal obesity, arterial hypertension, hyperinsulinaemia, hyperglycaemia, type 2 diabetes and the dyslipidaemia characteristically appearing with insulin resistance. This dyslipidaemia, also known as the atherogenic lipoprotein profile, is characterised by moderately elevated non-esterified fatty acids, elevated very low density lipoprotein (VLDL) triglyceride rich particles, high Apo B levels, low high density lipoprotein (HDL) levels associated with low apoAI particle levels and high Apo B levels in the presence of small, dense, low density lipoproteins (LDL) particles, phenotype B.

The compounds of the present invention are expected to be useful in treating patients with combined or mixed hyperlipidemias or various degrees of hypertriglyceridemias and postprandial dyslipidemia with or without other manifestations of the metabolic syndrome.

Treatment with the present compounds is expected to lower the cardiovascular morbidity and mortality associated with atherosclerosis due to their antidyslipidaemic as well as antiinflammatory properties. The cardiovascular disease conditions include macro-angiopathies of various internal organs causing myocardial infarction, congestive heart failure, cerebrovascular disease and peripheral arterial insufficiency of the lower extremities. Because of their insulin sensitizing effect the compounds of formula I are also expected to prevent or delay the development of type 2 diabetes from the metabolic syndrome and diabetes of pregnancy. Therefore the development of long-term complications associated with chronic hyperglycaemia in diabetes mellitus such as the micro-angiopathies causing renal disease, retinal damage and peripheral vascular disease of the lower limbs are expected to be delayed. Furthermore the compounds may be useful in treatment of various conditions outside the cardiovascular system whether or not associated with insulin resistance, like polycystic ovarian syndrome, obesity, cancer and states of inflammatory disease including neurodegenerative disorders such as mild cognitive impairment, Alzheimer's disease, Parkinson's disease and multiple sclerosis.

The compounds of the present invention are expected to be useful in controlling glucose levels in patients suffering from type 2 diabetes.

The present invention provides a method of treating or preventing dyslipidemias, the insulin resistance syndrome and/or metabolic disorders (as defined above) comprising the administration of a compound of formula I to a mammal (particularly a human) in need thereof.

The present invention provides a method of treating or preventing type 2 diabetes comprising the administration of an effective amount of a compound of formula I to a mammal (particularly a human) in need thereof.

In a further aspect the present invention provides the use of a compound of formula I as a medicament.

In a further aspect the present invention provides the use of a compound of formula I in the manufacture of a medicament for the treatment of insulin resistance and/or metabolic disorders.

Combination Therapy

The compounds of the invention may be combined with other therapeutic agents that are useful in the treatment of disorders associated with the development and progress of atherosclerosis such as hypertension, hyperlipidaemias, dyslipidaemias, diabetes and obesity. The compounds of the invention may be combined with another therapeutic agent that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of metabolic syndrome or type 2 diabetes and its associated complications, these include biguanide drugs, for example metformin, phenformin and buformin, insulin (synthetic insulin analogues, amylin) and oral antihyperglycemics (these are divided into prandial glucose regulators and alpha-glucosidase inhibitors). An example of an alpha-glucosidase inhibitor is acarbose or voglibose or miglitol. An example of a prandial glucose regulator is repaglinide or nateglinide.

In another aspect of the invention, the compound of formula I, or a pharmaceutically acceptable salt thereof, may be administered in association with a PPAR modulating agent. PPAR modulating agents include but are not limited to a PPAR alpha and/or gamma and/or delta agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art. These include the compounds described in WO 01/12187, WO 01/12612, WO 99/62870, WO 99/62872, WO 99/62871, WO 98/57941, WO 01/40170, WO 04/000790, WO 04/000295, WO 04/000294, WO 03/051822, WO 03/051821, WO 02/096863, WO 03/051826, WO 02/085844, WO 01/040172, J Med Chem, 1996, 39, 665, Expert Opinion on Therapeutic Patents, 10 (5), 623-634 (in particular the compounds described in the patent applications listed on page 634) and J Med Chem, 2000, 43, 527 which are all incorporated herein by reference. Particularly a PPAR alpha and/or gamma and/or delta agonist refers to muraglitazar (BMS 298585), rivoglitazone (CS-011), netoglitazone (MCC-555), balaglitazone (DRF-2593, NN-2344), clofibrate, fenofibrate, bezafibrate, gemfibrozil, ciprofibrate, pioglitazone, rosiglitazone, AVE-0847, AVE-8134, CLX-0921, DRF-10945, DRF-4832, LY-518674, LY-818, LY-929, 641597, GW-590735, GW-677954, GW-501516, MBX-102, ONO-5129, KRP-101, R-483 (BM131258), TAK-559 or TAK-654. Particularly a PPAR alpha and/or gamma and/or delta agonist refers to tesaglitazar ((S)-2-ethoxy-3-[4-(2-{4-methanesulphonyl-oxyphenyl}ethoxy)phenyl]propanoic acid) and pharmaceutically acceptable salts thereof.

In addition the combination of the invention may be used in conjunction with a sulfonylurea for example: glimepiride, glibenclamide (glyburide), gliclazide, glipizide, gliquidone, chloropropamide, tolbutamide, acetohexamide, glycopyramide, carbutamide, glibonuride, glisoxepid, glybuthiazole, glibuzole, glyhexamide, glymidine, glypinamide, phenbutamide, tolcylamide and tolazamide. Preferably the sulfonylurea is glimepiride or glibenclamide (glyburide). More preferably the sulfonylurea is glimepiride. Therefore the present invention includes administration of a compound of the present invention in conjunction with one, two or more existing therapies described in this paragraph. The doses of the other existing therapies for the treatment of type 2 diabetes and its associated complications will be those known in the art and approved for use by regulatory bodies for example the FDA and may be found in the Orange Book published by the FDA. Alternatively smaller doses may be used as a result of the benefits derived from the combination.

The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin selected from the group consisting of atorvastatin, bervastatin, cerivastatin, dalvastatin, fluvastatin, itavastatin, lovastatin, mevastatin, nicostatin, nivastatin, pravastatin and simvastatin, or a pharmaceutically acceptable salt, especially sodium or calcium, or a solvate thereof, or a solvate of such a salt. A particular statin is atorvastatin, or a pharmaceutically acceptable salt, solvate, solvate of such a salt or a prodrug thereof. A more particular statin is atorvastatin calcium salt. A particularly preferred statin is, however, a compound with the chemical name (E)-7-[4-(4-fluorophenyl)-6-isopropyl-2-[methyl(methylsulfonyl)-amino]-pyrimidin-5-yl](3R,5S)-3,5-dihydroxyhept-6-enoic acid, [also known as (E)-7-[4-(4-fluorophenyl)-6-isopropyl-2-[N-methyl-N-(methylsulfonyl)-amino]pyrimidin-5-yl](3R,5S)-3,5-dihydroxyhept-6-enoic acid] or a pharmaceutically acceptable salt or solvate thereof, or a solvate of such a salt. The compound (E)-7-[4-(4-fluorophenyl)-6-isopropyl-2-[methyl-(methylsulfonyl)-amino]-pyrimidin-5-yl](3R,5S)-3,5-dihydroxyhept-6-enoic acid, and its calcium and sodium salts are disclosed in European Patent Application, Publication No. EP-A-0521471, and in Bioorganic and Medicinal Chemistry, (1997), 5(2), 437-444. This latter statin is now known under its generic name rosuvastatin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor).

Suitable compounds possessing IBAT inhibitory activity have been described, see for instance the compounds described in WO 93/16055, WO 94/18183, WO 94/18184, WO 96/05188, WO 96/08484, WO 96/16051, WO 97/33882, WO 98/07449, WO 98/03818, WO 98/38182, WO 99/32478, WO 99/35135, WO 98/40375, WO 99/35153, WO 99/64409, WO 99/64410, WO 00/01687, WO 00/47568, WO 00/61568, WO 00/62810, WO 01/68906, DE 19825804, WO 00/38725, WO 00/38726, WO 00/38727, WO 00/38728, WO 00/38729, WO 01/68906, WO 01/66533, WO 02/32428, WO 02/50051, EP 864 582, EP489423, EP549967, EP573848, EP624593, EP624594, EP624595 and EP624596 and the contents of these patent applications are incorporated herein by reference.

Particular classes of IBAT inhibitors suitable for use in the present invention are benzothiepines, and the compounds described in the claims, particularly claim 1, of WO 00/01687, WO 96/08484 and WO 97/33882 are incorporated herein by reference. Other suitable classes of IBAT inhibitors are the 1,2-benzothiazepines, 1,4-benzothiazepines and 1,5-benzothiazepines. A further suitable class of IBAT inhibitors is the 1,2,5-benzothiadiazepines.

One particular suitable compound possessing IBAT inhibitory activity is (3R,5R)-3-butyl-3-ethyl-1,1-dioxido-5-phenyl-2,3,4,5-tetrahydro-1,4-benzothiazepin-8-ylβ3-D-glucopyranosiduronic acid (EP 864 582). Other suitable IBAT inhibitors include one of:

According to an additional further aspect of the present invention there is provided a combination treatment comprising the administration of an effective amount of a compound of the formula I, or a pharmaceutically acceptable salt, solvate, solvate of such a salt or a prodrug thereof, optionally together with a pharmaceutically acceptable diluent or carrier, with the simultaneous, sequential or separate administration one or more of the following agents selected from:

a CETP (cholesteryl ester transfer protein) inhibitor, for example those referenced and described in WO 00/38725 page 7 line 22-page 10, line 17 which are incorporated herein by reference;

a cholesterol absorption antagonist for example azetidinones such as SCH 58235 and those described in U.S. Pat. No. 5,767,115 which are incorporated herein by reference;

a MTP (microsomal transfer protein) inhibitor for example those described in Science, 282, 751-54, 1998 which are incorporated herein by reference;

a nicotinic acid derivative, including slow release and combination products, for example, nicotinic acid (niacin), acipimox and niceritrol;

a phytosterol compound for example stanols;

probucol;

an anti-obesity compound for example orlistat (EP 129,748) and sibutramine (GB 2,184,122 and U.S. Pat. No. 4,929,629);

an antihypertensive compound for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, an AT-1 blocker, a saluretic, a diuretic or a vasodilator;

a CB1 antagonist or inverse agonist for example as described in WO01/70700 and EP 65635;

a Melanin concentrating hormone (MCH) antagonist;

a PDK inhibitor; or

modulators of nuclear receptors for example LXR, FXR, RXR, and RORalpha;

or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Particular ACE inhibitors or pharmaceutically acceptable salts, solvates, solvate of such salts or a prodrugs thereof, including active metabolites, which can be used in combination with a compound of formula I include but are not limited to, the following compounds: alacepril, alatriopril, altiopril calcium, ancovenin, benazepril, benazepril hydrochloride, benazeprilat, benzoylcaptopril, captopril, captopril-cysteine, captopril-glutathione, ceranapril, ceranopril, ceronapril, cilazapril, cilazaprilat, delapril, delapril-diacid, enalapril, enalaprilat, enapril, epicaptopril, foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril and zofenoprilat. Preferred ACE inhibitors for use in the present invention are ramipril, ramiprilat, lisinopril, enalapril and enalaprilat. More preferred ACE inhibitors for uses in the present invention are ramipril and ramiprilat.

Preferred angiotensin II antagonists, pharmaceutically acceptable salts, solvates, solvate of such salts or a prodrugs thereof for use in combination with a compound of formula I include, but are not limited to, compounds: candesartan, candesartan cilexetil, losartan, valsartan, irbesartan, tasosartan, telmisartan and eprosartan. Particularly preferred angiotensin II antagonists or pharmaceutically acceptable derivatives thereof for use in the present invention are candesartan and candesartan cilexetil.

Therefore in an additional feature of the invention, there is provided a method for for the treatment of type 2 diabetes and its associated complications in a warm-blooded animal, such as man, in need of such treatment which comprises administering to said animal an effective amount of a compound of formula I, or a pharmaceutically acceptable salt, solvate, solvate of such a salt or a prodrug thereof in simultaneous, sequential or separate administration with an effective amount of one the other compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt, solvate, solvate of such a salt or a prodrug thereof in simultaneous, sequential or separate administration with an effective amount of one the other compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt, solvate, solvate of such a salt or a prodrug thereof, and one of the other compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt, solvate, solvate of such a salt or a prodrug thereof, and one of the other compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt, solvate, solvate of such a salt or a prodrug thereof, in a first unit dosage form;

b) one of the other compounds described in this combination section or a pharmaceutically acceptable salt, solvate, solvate of such a salt or a prodrug thereof; in a second unit dosage form; and

c) container means for containing said first and second dosage forms.

According to a further aspect of the present invention there is provided a kit comprising:

a) a compound of formula I, or a pharmaceutically acceptable salt, solvate, solvate of such a salt or a prodrug thereof, together with a pharmaceutically acceptable diluent or carrier, in a first unit dosage form;

b) one of the other compounds described in this combination section or a pharmaceutically acceptable salt, solvate, solvate of such a salt or a prodrug thereof, in a second unit dosage form; and

c) container means for containing said first and second dosage forms.

According to another feature of the invention there is provided the use of a compound of the formula I, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the the treatment of metabolic syndrome or type 2 diabetes and its associated complications in a warm-blooded animal, such as man.

According to another feature of the invention there is provided the use of a compound of the formula I, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt, solvate, solvate of such a salt or a prodrug thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

### EXAMPLES

¹H NMR and ¹³C NMR measurements were performed on a Varian Mercury 300 or Varian UNITY plus 400, 500 or 600 spectrometers, operating at ¹H frequencies of 300, 400, 500 and 600 MHz, respectively, and at ¹³C frequencies of 75, 100, 125 and 150 MHz, respectively. Measurements were made on the delta scale (δ).

Unless otherwise stated, chemical shifts are given in ppm with the solvent as internal standard

Abbreviations

| | |
|---|---|
| DMSO | dimethyl sulfoxide |
| EtOAc | ethyl acetate |
| DMF | N,N-dimethylformamide |
| THF | tetrahydrofuran |
| MeCN | acetonitrile |
| MeOH | methanol |
| TFA | trifluoroacetic acid |
| NH₄OAc | ammonium acetate |
| t | triplet |
| s | singlet |
| d | doublet |
| q | quartet |
| m | multiplet |
| bs | broad singlet |

Starting Materials and Intermediates

### Compound A. S-(4-Cyanobenzyl)ethanethioate

To a stirred solution of 4-(bromomethyl)benzonitrile (4.00 mmol, 784 mg) and thioacetic acid (4.20 mmol, 320 mg) in MeOH (8 mL) was added dropwise triethylamine (4.20 mmol, 425 mg). After cooling, the resulting solution was used in the next reaction step.

### Compound B. S-{2-[4-(Dimethylamino)phenyl]ethyl}ethanethioate

2-[4-(Dimethylamino)phenyl]ethanol (4.00 mmol, 661 mg) and triethylamine (4.80 mmol, 486 mg) were dissolved in DCM (15 mL) and cooled in an ice-bath. Methanesulfonyl chloride (4.40 mmol, 504 mg) was added in portions and the ice-bath was removed. After 1.5 h water was added. The phases were separated. The organic phase was filtered through MgSO₄ and evaporated to dryness. The crude mesylate was dissolved in MeOH (8 mL). To this solution was added triethylamine (4.20 mmol, 425 mg) and thioacetic acid (4.20 mmol, 320 mg). After cooling, the resulting solution was used in the next reaction step.

### Compound C. S-[2-(2-Thienyl)ethyl]ethanethioate

A solution of the title compound was prepared from 2-(2-thienyl)ethyl methanesulfonate using the procedure described for compound A,

### Compound D. S-[2-(2-fluorophenyl)ethyl]ethanethioate

A solution of the title compound was prepared from 2-(2-fluorophenyl)ethanol using the procedure described for compound B.

### Compound E. S-[2-(3-Methoxyphenyl)ethyl]ethanethioate

A solution of the title compound was prepared from 2-(3-methoxyphenyl)ethanol using the procedure described for compound B.

### Compound F. Methyl 2-chloro-3-[4-(2-{4-[(methylsufonyl)oxy]phenoxy}ethyl)phenyl]propanoate

### (i) Methyl 2-chloro-3-[4-(2-hydroxyethyl)phenyl]propanoate

2-(4-Aminophenyl)ethanol (11 g, 8 mmol) and 32 ml conc HCl was dissolved in acetone and cooled to 0° C. Sodium nitrite (5.6 g, 81 mmol) in 20 ml water was added dropwise. The temperature was kept under 0° C. After one hour, methyl acrylate (70 g, 808 mmol) and CuI (1.6 g, 8 mmol) were added (<0° C.). The reaction mixture was stirred at room temperature overnight. The solvent was evaporated and water was added. The water phase was extracted three times with EtOAc, the organic phases were pooled and washed with water, dried (MgSO₄) and evaporated under reduced pressure. The crude product was purified by flash chromatography using a 65:35 mixture of EtOAc and heptane as eluent. Further purification by preparative HPLC (using a gradient of CH₃CN/5% CH₃CN-waterphase containing 0.1M NH₄OAc as eluent) gave 9.7 g product (yield 49%) as an oil

¹HNMR (400 MHz, CDCl₃): 2.84 (t, 3H), 3.15 (dd, 1H), 3.35 (dd, 1H), 3.75 (s, 3H), 3.84 (t, 3H), 4.43 (t, 1H), 7.17 (d, 4H)

### (ii) Methyl 3-(4-{2-[4-(benzyloxy)phenoxy]ethyl}phenyl)-2-chloropropanoate

Triphenylphosphine (2.4 g, 9 mmol) was added to a solution of methyl 2-chloro-3-[4-(2-hydroxyethyl)phenyl]propanoate (2.1 g, 8.5 mmol) and 4-(benzyloxy)phenol (1.7 g, 8 mmol) in 20 ml toluene under nitrogen atmosphere. The solution was warmed to 55° C. and diisopropyl azodicarboxylate (1.8 g, 9 mmol) was added. The reaction mixture was stirred at 55° C. overnight. The mixture was allowed to cool and the solvent was evaporated under reduced pressure. The crude product was purified by flash chromatography using a 80:20 mixture of heptane and EtOAc as eluent to yield 2.28 g of the desired product (yield 61%) as colourless crystals.

¹HNMR (400 MHz, CDCl₃): 3.05 (t, 2H), 3.16 (dd, 1H), 3.36 (dd, 1H), 3.75 (s, 3H), 4.12 (t, 2H), 4.45 (t, 1H), 5.01 (s, 2H), 6.82 (m, 2H), 6.90 (m, 2H), 7.13-7.27 (m, 4H), 7.29-7.47 (m, 5H).

### (iii) Methyl 2-chloro-3-{4-[2-(4-hydroxyphenoxy)ethyl]phenyl}propanoate

Methyl 3-(4-{2-[4-(benzyloxy)phenoxy]ethyl}phenyl)-2-chloropropanoate (1.0 g, 2.4 mmol) and dimethyl sulfide (0.9 g, 14 mmol) was dissolved in 60 ml CH₂Cl₂. Boron trifluoride etherate (2.0 g, 14 mmol) was added droppwise to the stirred solution. The reaction mixture was stirrred for two days at room temperature. Another equivalent (0.4 g, 2.87 mmol) boron trifluoride etherate was added and the stirring was continued overnight.

Water was added. The phases were separated and the aqueous phase was extracted twice with CH₂Cl₂. The organic phases were pooled, washed (water, brine), dried (Na₂SO₄) and evaporated under reduced pressure. Further purification by preparative HPLC using a gradient of CH₃CN/5% CH₃CN-waterphase containing 0.1M NH₄OAc gave 0.55 g of the desired product (yield 52%) as an oil.

¹HNMR (400 MHz, CDCl₃): 3.04 (t, 2H), 3.16 (dd, 1H), 3.35 (dd, 1H), 3.75 (s, 3H), 4.10 (t, 2H), 4.40 (t, 1H), 6.75 (m, 4H), 7.12-7.29 (m, 4H).

### (iv) Methyl 2-chloro-3-[4-(2-{4-[(methylsulfonyl)oxy]phenoxy}ethyl)phenyl]propanoate

Methyl 2-chloro-3-{4-[2-(4-hydroxyphenoxy)ethyl]phenyl}propanoate (334 mg, 1.0 mmol) and triethylamine (303 mg, 3.0 mmol) was dissolved in 20 ml dichloromethane and cooled to −20° C. under nitrogen atmosphere. Methanesulfonyl chloride (114 mg, 1.0 mmol) was added dropwise. The mixture was allowed to reach room temperature. After 2 hours dichlormethane was added, the mixture was washed (water, brine), dried (Na₂SO₄) and evaporated under reduced pressure to yield 394 mg pure product (yield 96%).

¹HNMR (400 MHz, CDCl₃): 3.02-3.11 (m,5H), 3.15 (dd, 1H), 3.35 (dd, 1H), 3.74 (s, 3H), 4.14 (t, 2H), 4.44 (t, 1H), 5.29 (s, 2H), 6.88 (d, 2H), 7.14-7.25 (m, 6H).

### EXAMPLES

### Example 1

### 2-[(4-Cyanobenzyl)thio]-3-[4-(2-{4-[(methylsulfonyl)oxy]phenoxy}ethyl)-phenyl]propanoic acid

The reaction was performed under an argon atmosphere. To 0.80 mL of a stirred solution of compound A (0.40 mmol) in MeOH was added sodium methane thiolate (0.80 mmol, 56 mg) in MeOH (0.20 mL). After one hour of stirring, compound F (0.48 mmol, 200 mg) in MeCN was added. After 16 h of stirring, the mixture was evaporated to dryness using a vacuum centrifuge. The residual crude product was dissolved in 0.5 M LiOH solution (THF/water 7:1, 0.50 mL) and stirred for 20 hours. After acidification with 12 M HCl (100 μL) the stirring was continued for one hour. The crude product was filtered through a Teflon™ filter and purified using preparative HPLC (C8-column, gradient of 0.2% TFA/MeCN) to give 24 mg of the title compound. ¹H-NMR (400 MHz, CDCl₃): 2.80-2.88 (m, 1H), 3.06 (t, J=6.9 Hz, 2H), 3.10 (s, 3H), 3.10-3.18 (m, 1H), 3.30 (t, J=7.7 Hz, 1H), 3.77-3.93 (m, 2H), 4.15 (t, J=6.9 Hz, 2H), 6.85-6.90 (m, 2H), 7.00-7.06 (m, 2H), 7.14-7.20 (m, 4H), 7.33-7.38 (m, 2H), 7.50-7.55 (m, 2H)

### Example 2

### 2-({2-[4-(Dimethylamino)phenyl]ethyl}thio)-3-[4-(2-{4-[(methylsulfonyl)oxy]-phenoxy}ethyl)phenyl]propanoic acid

The title compound (yield 6 mg) was prepared from compound B and F using the procedure described for example 1. ¹H-NMR (400 MHz, CDCl₃): 2.70-2.96 (m, 5H), 2.90 (s, 6H), 3.05 (t, J=7.0 Hz, 2H), 3.10 (s, 3H), 3.13-3.20 (m, 2H), 3.47-3.53 (m, 2H), 4.12 (t, J=7.0 Hz, 2H), 6.66-6.72 (m, 2H), 6.83-6.89 (m, 2H), 7.00-7.05 (m, 2H), 7.13-7.20 (m, 4H)

### Example 3

### 3-[4-(2-{4-[(Methylsulfonyl)oxy]phenoxy}ethyl)phenyl]-2-{[2-(2-thienyl)ethyl]thio}propanoic acid

The title compound (yield 3 mg) was prepared from compound C and P using the procedure described for example 1. ¹H-NMR (400 MHz, CDCl₃): 2.85-3.00 (m, 4H), 3.02-3.13 (m, 6H), 3.15-3.22 (m, 1H), 3.50-3.56 (m, 1H), 4.13 (t, J=7.0 Hz, 2H), 6.77-6.80 (m, 1H), 6.84-6.87 (m, 2H), 6.87-6.92 (m, 1H), 7.10-7.13 (m, 1H), 7.15-7.19 (m, 6H)

### Example 4

### 2-{[2-(2-Fluorophenyl)ethyl]thio}-3-[4-(2-{4-[(methylsulfonyl)oxy]phenoxy}-ethyl)phenyl]propanoic acid

The title compound (yield 2 mg) was prepared from compound D and F using the procedure described for example 1. ¹H-NMR (400 MHz, CDCl₃): 2.83-2.94 (m, 4H), 2.94-3.00 (m, 1H), 3.05 (t, J=7.1 Hz, 2H), 3.09 (s, 3H), 3.14-3.22 (m, 1H), 3.51-3.57 (m, 1H), 4.13 (t, J=7.1 Hz, 2H), 6.83-6.88 (m, 2H), 6.96-7.02 (m, 1H), 7.03-7.06 (m, 1H), 7.11-7.22 (m, 8H)

### Example 5

### 2-{[2-(3-Methoxyphenyl)ethyl]thio}-3-[4-(2-{4-[(methylsulfonyl)oxy]phenoxy}-ethyl)phenyl]propanoic add

The title compound (yield 16 mg) was prepared from compound E and F using the procedure described for example 1. ¹H-NMR (400 MHz, CDCl₃): 1.99-2.19 (m, 5H), 2.30 (t, J=6.9 Hz, 2H), 2.34 (s, 3H), 2.35-2.41 (m, 1H), 2.69-2.75 (m, 1H), 3.00 (s, 3H), 3.37 (t, J=6.9 Hz), 5.92-6.04 (m, 6H), 6.37-6.48 (m, 4H), 6.52-6.58 (m, 2H)

### Example 6

### 2-{[2-(4-Hydroxyphenyl)ethyl]sulfinyl}-3-[4-(2-{4-[(methylsulfonyl)oxy]phenoxy}-ethyl)phenyl]propanoic acid

### a) 2-[4-(Benzyloxy)phenyl]ethanol

2-(4-Hydroxyphenyl)ethyl alcohol (30.5 g, 0.22 mol), benzyl bromide (39.6 g, 0.23 mol) and potassium carbonate (33.5 g, 0.24 mol) were mixed and boiled under reflux for 4 hours. The reaction was left at room temperature for 12 hours. The solvent was evaporated and the residue was dissolved in water and chloroform. The phases were separated and the water phase was extracted one more time. The organic phases were pooled, dried (MgSO4) and evaporated to give 47 g of the desired product (93% yield).

### b) 2-[4-(Benzyloxy phenyl]ethyl methanesulfonate

2-[4-(Benzyloxy)phenyl]ethanol (22 g, 96 mmol), triethylamine (29 g, 289 mmol) and 200 ml of dichloromethane were mixed and cooled to −20° C. under a nitrogen atmosphere. Mesyl chloride (11 g, 96 mmol) was added dropwise. The reaction was stirred until it reached room temperature. The mixture was diluted with dichloromethane, washed twice with water, dried (MgSO₄) and evaporated to give 31 g of the desired product as brown crystals in quantitative yield.

¹HNMR (400 MHz, CDCl₃): 2.82 (s, 3H), 2.98 (t, 2H), 4.37 (t, 2H), 5.05 (s, 2H), 6.91-6.96 (m, 2H), 7.12-7.17 (m, 2H), 7.28-7.46 (m, 5H).

### c) S—{2-[4-(benzyloxy)phenyl]ethyl}ethanethioate

Cesium carbonate (33 g, 101 mmol) was added to a solution of thioacetic acid (8.47 g, 111 mmol) in 60 ml methanol. After 30 minutes the solvent was evaporated and 160 ml DMF was added. 2-[4-(Benzyloxy)phenyl]ethyl methanesulfonate (31 g, 101 mmol) was added drop wise. The mixture was stirred overnight at room temperature. Diethyl ether was added (˜1 liter). The organic phase was washed 5 times with water and once with brine, dried (MgSO₄) and evaporated. The crude was further purified by flash chromatography using toluene as eluent to give 19 g of the desired product (65% yield).

¹HNMR (400 MHz, CDCl₃): 2.35 (s, 3H), 2.84 (t, 2H), 3.11 (t, 2H), 5.07 (s, 2H), 6.92-6.97 (m, 2H), 7.13-7.18 (m, 2H), 7.33-7.47 (m, 5H).

### d) Methyl 2-chloro-3-[4-(2-hydroxyethyl)phenyl]propanoate

4-Aminophenethyl alcohol (15 g, 109 mmol), 200 ml acetone and 43 ml hydrochloric acid were mixed and cooled on an ice bath. Sodium nitrite (7.5 g, 109 mmol) dissolved in 22 ml water was added and the temperature was kept at ˜0° C. After 1 h methyl acrylate (94.1 g, 1093 mmol) was added and then Cu(I) I (2.08 g, 10.9 mmol) in portions (still at 0° C.). The reaction was stirred for ˜1.5 h at 0° C. and then overnight at room temperature. The acetone was evaporated and water was added. The water phase was extracted three times with ethyl acetate. The organic phase was washed with water, dried (MgSO₄) and evaporated. The crude product was filtered through a silica column (dichloromethane/methanol (99:1)). This crude product was put on another silica column using toluene/ethyl acetate (50:50) as eluent to give 19 g of the desired product plus a by-product. Further purification on preparative HPLC using a gradient of CH₃CN/10% CH₃CN-waterphase containing 0.1M ammoniumacetate, 20% CH₃CN to 100% CH₃CN in 50 min, gave 12.7 g pure product (46% yield) as a light yellow oil.

¹HNMR (400 MHz, CDCl₃): 2.84 (t, 2H), 3.15 (dd, 1H), 3.35 (dd, 1H), 3.75 (s, 3H), 3.84 (t, 2H), 4.43 (t, 1H), 7.15-7.18 (m, 4H).

### e) Methyl 3-(4-{2-[4-(benzyloxy)phenoxy]ethyl}phenyl)-2-chloropropanoate

Methyl 2-chloro-3-[4-(2-hydroxyethyl)phenyl]propanoate (12.2 g, 50.6 mmol) and 4-(benzyloxy)phenol (10.1 g, 50.6 mmol) was dissolved in 350 ml dry toluene. Triphenylphosphine (14.6 g, 55.6 mmol) was added. The mixture was heated to 55° C. and diisopropyl azodicarboxylate (11.2 g, 55.6 mmol) was added. The reaction mixture was stirred overnight at 55° C. The reaction was followed on thin layer chromatography (heptane/ethyl acetate (80:20)). The solvent was evaporated and diethyl ether was added. The triphenylphosphine oxide was filtered off, the solvent was evaporated and the residue was purified by flash chromatography using toluene as eluent to yield 12.3 g of the desired product (57% yield) as white crystals.

¹HNMR (400 MHz, CDCl₃): 3.05 (t, 2H), 3.16 (dd, 1H), 3.36 (dd, 1H), 3.75 (s, 3H), 4.11 (t, 2H), 4.45 (t, 1H), 5.02 (s, 2H), 6.80-6.84 (m, 2H), 6.88-6.91 (m, 2H),

7.14-7.28 (m, 4H), 7.29-7.44 (m, 5H).

### f) Methyl 2-chloro-3-{4-[2-(4-hydroxyphenoxy)ethyl]phenyl}propanoate

Methyl 3-(4-{2-[4-(benzyloxy)phenoxy]ethyl}phenyl)-2-chloropropanoate (12.3 g, 28.9 mmol) and dimethyl sulfide (8.99 g, 144.7 mmol) were dissolved in 450 ml dichloromethane. Boron trifluoride etherate (20.5 g, 144.7 mmol) was added dropwise. The mixture was stirred for two days at room temperature. Water was added and the phases were separated. The water phase was extracted twice with dichloromethane. The organic phases were pooled and washed (water, brine), dried and evaporated. The crude product was further purified by flash chromatography using an 80:20 mixture of toluene/ethyl acetate as eluent to yield 8.9 g (91% yield) of the desired product after freeze-drying.

¹HNMR (400 MHz, CDCl₃): 3.04 (t, 2H), 3.15 (dd, 1H), 3.35 (dd, 1H), 3.75 (s, 3H), 4.10 (t, 2H), 4.45 (t, 1H), 6.72-6.79 (m, 4H), 7.15-7.28 (m, 4H).

### g) Methyl 2-chloro-3-[4-(2-{4-[(methylsulfonyl)oxy]phenoxy}ethyl)phenyl]propanoate

To a solution of methyl 2-chloro-3-{4-[2-(4-hydroxyphenoxy)ethyl]phenyl}propanoate (8.9 g, 26.6 mmol) and triethylamine (8.07 g, 79.7 mmol) in 250 ml dichloromethane cooled to −20° C. under nitrogen atmosphere was added methanesulfonyl chloride (3.05 g, 26.6 mmol) drop wise. The reaction was stirred until it reached room temperature.

Dichloromethane was added and the organic phase was washed twice with water and once with brine, dried (MgSO₄) and evaporated. The crude product was further purified by flash chromatography using a 99.5:0.5 mixture of dichloromethane/methanol as eluent to give 10.6 g (97% yield) of the desired product.

¹HNMR (400 MHz, CDCl₃): 3.07 (t, 2H), 3.09 (s, 3H), 3.15 (dd, 1H), 3.35 (dd, 1H), 3.74 (s, 3H), 4.14 (t, 2H), 4.44 (t, 1H), 6.87-6.89 (m, 2H), 7.16-7.25 (m, 6H).

### h) Methyl 2-({2-[4-(benzyloxy)phenyl]ethyl}thio)-3-[4-(2-{4-[(methylsulfonyl)oxy]-phenoxy}ethyl)phenyl]propanoate

S-{2-[4-(benzyloxy)phenyl]ethyl}ethanethioate (6.70 g, 23.4 mmol) was dissolved in 43 ml methanol under argon atmosphere. To this slurry was added sodium thiomethoxide (1.64 g, 23.4 mmol) dissolved in 22 ml methanol. The reaction mixture was stirred for 30 minutes under an argon atmosphere. Half of the solvent volume was evaporated.

Methyl 2-chloro-3-[4-(2-{4-[(methylsulfonyl)oxy]phenoxy}ethyl)phenyl]propanoate (9.65 g, 23.4 mmol) dissolved in 18 ml dry DMF was added to the concentrated reaction mixture and stirred for 30 min under an argon atmosphere. The solvent was evaporated and the residue was dissolved in ethyl acetate. The organic phase was extracted five times with water and once with brine, dried (MgSO₄) and evaporated. The crude product was further purified by flash chromatography using a 90:10 mixture of toluene/ethyl acetate as eluent to give 13.7 g of the desired product (95% yield).

¹HNMR (400 MHz, CDCl₃): 2.73-2.86 (m, 4H), 2.92 (dd, 1H), 3.06 (t, 2H), 3.09 (s, 3H), 3.18 (dd, 1H), 3.48-3.52 (m, 1H), 3.67 (s, 3H), 4.13 (t, 2H), 5.04 (s, 2H), 6.86-6.91 (m, 4H), 7.05-7.09 (m, 2H), 7.11-7.22 (m, 6H), 7.30-7.43 (m, 5H).

### i) Methyl 2-{[2-(4-hydroxyphenl)ethyl thio}-3-[4-(2-{4-[(methylsulfonyl)oxy]phenoxy}-ethyl)phenyl]propanoate

Methyl 2-({2-[4-(benzyloxy)phenyl]ethyl}thio)-3-[4-(2-{4-[(methylsulfonyl)oxy]phenoxy}-ethyl)phenyl]propanoate (13.79 g, 22.2 mmol) and dimethyl sulfide (6.90 g, 111 mmol) was dissolved in 320 ml dichloromethane. Boron trifluoride etherate (15.76 g, 111 mmol) was added dropwise. The reaction was stirred at room temperature for 2 days. Water was added and the phases were separated. The water phase was washed twice with dichloromethane. The organic phases were pooled and washed (water, brine), dried (MgSO₄) and evaporated. The crude product was purified by flash chromatography using a 30:70 mixture of ethyl acetate/toluene as eluent to give 10.35 g of the desired product (87% yield).

¹HNMR (400 MHz, CDCl₃): 2.71-2.84 (m, 4H), 2.91 (dd, 1H), 3.06 (t, 2H), 3.10 (s, 3H), 3.17 (dd, 1H), 3.47-3.51 (m, 1H), 3.68 (s, 3H), 4.13 (t, 2H), 6.71-6.74 (m, 2H), 6.86-6.89 (m, 2H), 6.98-7.01 (m, 2H), 7.12-7.27 (m, 6H).

### j) (+)-Methyl 2-{[2-(4-hydroxyphenyl)ethyl]thio}-3-[4-(2-{4-[(methylsulfonyl)oxy]-phenoxy}ethyl)phenyl]propanoate and (−)-Methyl 2-{[2-(4-hydroxphenyl)ethyl]thio}-3-[4-(2-{4-[(methylsulfonyl)oxy]phenoxy}ethyl)phenyl]propanoate

Preparative chiral chromatography of methyl 2-{[2-(4-hydroxyphenyl)ethyl]thio}-3-[4-(2-{4-[(methylsulfonyl)oxy]phenoxy}ethyl)phenyl]propanoate Chiralpak AD (250×50 mm i.d.), 100% ethanol as mobile phase, flow rate 80 ml/min, UV-detection at 225 in, ALP 670 nm. Methyl 2-{[2-(4-hydroxyphenyl)ethyl]thio}-3-[4-(2-{4-[(methylsulfonyl)oxy]phenoxy}-ethyl)phenyl]propanoate (1.01 g) was dissolved in pure ethanol (10 mg/ml), 50-100 mg was loaded on the column. The separation of the two enantiomers was partial and therefore a middle fraction between the peaks was collected. The middle fractions were continuously evaporated to a proper volume which was re-injected, however, with an unknown sample concentration. Injections were made every 25 minutes when the second enantiomer's peak maximum had been passed. The optical rotation was monitored on-line with an ALP-detector: E1 rotated counter-clockwise (−), and E2 rotated clockwise (+).

E1: 462 mg (91%), ee>99%, [α]²⁰_{D}−37 (c 1, MeOH)

E2: 461 mg (91%), ee 97.1%, [α]²⁰_{D+}35 (c 1, MeOH)

Analytical conditions: Chiralpak AD (4.6×250 mm), 100% ethanol, 0.5 ml/min, 225 nm. k′₁: 2.35, k′₂: 3.27, α: 1.39.

¹HNMR (400 MHz, CDCl₃): 2.71-2.83 (m, 4H), 2.91 (dd, 1H), 3.05 (t, 2H), 3.10 (s, 3H), 3.17 (dd, 1H), 3.46-3.50 (m, 1H), 3.67 (s, 3H), 4.13 (t, 2H), 6.70-6.74 (in 2H), 6.85-6.89 (m, 2H), 6.97-7.01 (m, 2H), 7.11-7.20 (m, 6H).)

### k) Methyl(2R-2-{[2-(hydroxyphenyl)ethyl]sulfinyl-3-[4-(2-{4-[(methylsulfonyl)oxy]-phenoxy}ethyl)phenyl]propanoate or Methyl(2S)-2-{[2-(4-hydroxyphenyl)ethyl]sulfinyl}-3-[4-(2-{4-[(methylsulfonyl)oxy]phenoxy}ethyl)phenyl]propanoate

Methyl(2S)-2-{[2-(4-hydroxyphenyl)ethyl]thio}-3-[4-(2-{4-[(methylsulfonyl)oxy]-phenoxy}ethyl)phenyl]propanoate or methyl(2R)-2-{[2-(4-hydroxyphenyl)ethyl]thio}-3-[4-(2-{4-[(methylsulfonyl)oxy]phenoxy}ethyl)phenyl]propanoate (188 mg, 0.35 mmol) was dissolved in 5 ml dichloromethane and cooled on an ice bath. 3-Chloroperbenzoic acid (87 mg, 0.35 mmol) was added in portions. The mixture was stirred for 2 h. 1M Na₂CO₃ was added. The phases were separated and the water phase was extracted twice with dichloromethane. The organic phases were pooled and washed with 1M Na₂CO₃-solution, dried (MgSO₄) and evaporated. The crude was further purified by preparative HPLC using a gradient of CH₃CN/5% CH₃CN-waterphase containing 0.1M ammoniumacetate, 20% CH₃CN to 100% CH₃CN in 45 minutes to give 149 mg product (77% yield) as a mixture of diasteriomers/epimers.

¹HNMR (400 MHz, CDCl₃) (diasteriomers): 2.85-3.08 (m, 6H), 3.10 (s, 3H), 3.21-3.34 (m, 2H), 3.66, 3.73 (s,s 3H), 3.73-3.77, 3.80-3.85 (m,m 1H), 4.13 (t, 2H), 6.74-6.78 (m, 2H), 6.85-6.89 (m, 2H), 7.01-7.06 (m, 2H), 7.12-7.22 (m, 6H).

### l) 2-{[2-(4-Hydroxypheny)ethyl]sulfinyl}-3-[4-(2-{4-[(methylsulfonyl)oxy]phenoxy}-ethyl]phenyl}propanoic acid

Methyl(2R)-2-{[2-(4-hydroxyphenyl)ethyl]sulfinyl}-3-[4-(2-{4-[(methylsulfonyl)oxy]-phenoxy}ethyl)phenyl]propanoate or methyl(2S)-2-{[2-(4-hydroxyphenyl)ethyl]sulfinyl}-3-[4-(2-{4-[(methylsulfonyl)oxy]phenoxy}ethyl)phenyl]propanoate (149 mg, 0.27 mmol) was dissolved in THF (4 ml) and cooled on an ice bath. Lithium hydroxide (0.7M, 1.36 mmol) (aq) was added dropwise. The mixture was stirred overnight at room temperature. Water was added and the solvent (THF) was removed by evaporation. The remaining water phase was acidified with 1M HCl and extracted twice with ethyl acetate. The organic phases were pooled and washed with brine, dried (MgSO4) and evaporated. The crude product was further purified by preparative HPLC using a gradient of CH₃CN/5% CH₃CN-waterphase containing 0.1M ammonium acetate, 20% CH₃CN to 100% CH₃CN in 30 minutes gave 89 mg product (61% yield) as a racemate

¹HNMR (500 MHz, CD₃OD): 2.90-3.32 (m, 11H), 3.84-3.91 (m, 1H), 4.17 (t, 2H), 6.70-6.75 (m, 2H), 6.92-6.95 (m, 2H), 7.04-7.09 (m, 2H), 7.18-7.26 (m, 6H).

### Example 7

### 3-{4-[2-(4-Benzoylphenoxy)ethyl]phenyl}-2-{[2-(4-hydroxyphenyl)ethyl]thio}propanoic acid

### a) 2-[4-(Benzyloxy)phenyl]ethanethiol

Sodium thiomethoxide (0.86 g, 12.2 mmol) was added to a solution of S-{2-[4-benzyloxy)phenyl]ethyl}ethanethioate (See Example 6 c) (3.5 g, 12.2 mmol) in 60 ml methanol. The mixture was stirred at room temperature for 30 minutes. 1M HCl was added and the water phase was extracted three times with dichloromethane. The organic phase was washed (brine), dried and evaporated to give 2.4 g of the desired product (80% yield).

¹HNMR (300 MHz, CDCl₃): 2.71-2.97 (m, 4H), 5.08 (s, 2H), 6.92-6.98 (m, 2H), 7.11-7.17 (m, 2H), 7.32-7.48 (m, 5H).

### b) Methyl 2-chloro-3-[4-(2-hydroxyethyl)phenyl]propanoate. See Example 6 step d

### c) Methyl 2-({2-[4-(benzyloxy)phenyl]ethyl}thio)-3-[4-(2-hydroxethyl)phenyl]propanoate

Methyl 2-chloro-3-[4-(2-hydroxyethyl)phenyl]propanoate (2 g, 8.24 mmol), 2-[4-(benzyloxy)phenyl]ethanethiol (2.4 g, 9.89 mmol), potassium carbonate (1.4 g, 9.89 mmol) and 100 ml DMF was stirred overnight at room temperature. The solvent was evaporated and the residue was dissolved in toluene. The organic phase was washed (water, brine), dried (MgSO₄) and evaporated. The crude was further purified by preparative hplc to give 1.55 g of the desired product (40% yield) as yellow oil.

¹HNMR (600 MHz, CDCl₃): 2.75-2.86 (m, 6H), 2.91 (dd, 1H), 3.17 (dd, 1H), 3.47-3.51 (m, 1H), 3.67 (s, 3H), 3.83 (t, 2H), 5.04 (s, 2H), 6.87-6.91 (m, 2H), 7.05-7.09 (m, 2H), 7.11-7.15 (m, 4H), 7.29-7.44 (m, 5H).

### d) Methyl 3-{4-[2-(4-benzoylphenoxy)ethyl]phenyl}-2-({2-[4-(benzyloxy)phenyl]ethyl}-thio)propanoate

Triphenylphosphine (120 mg, 0.46 mmol) was added to a solution of 4-benzoylphenol (82 mg, 0.42 mmol) and methyl 2-({2-[4-(benzyloxy)phenyl]ethyl}thio)-3-[4-(2-hydroxyethyl)-phenyl]propanoate (187 mg, 0.42 mmol) in 4 ml toluene. The mixture was heated to 55° C. and diisopropylazodicarboxylate (92 mg, 0.46 mmol) was added. The reaction was stirred at 55° C. for 24 h. The solvent was evaporated and the crude residue was purified by preparative hplc to give 225 mg of the desired product (83% yield).

¹HNMR (400 MHz, CDCl₃): 2.75-2.90 (m, 4H), 2.96 (dd, 1H), 3.12 (t, 2H), 3.21 (dd, 1H), 3.51-3.57 (m, 1H), 3.70 (s, 3H), 4.25 (t, 2H), 5.06 (s, 2H), 6.89-6.99 (m, 4H), 7.07-7.13 (m, 2H), 7.15-7.19 (m, 2H), 7.22-7.26 (m, 2H), 7.31-7.53 (m, 7H), 7.55-7.61 (m, 1H), 7.74-7.86 (m, 4H).

### e) Methyl 3-{4-[2-(4-benzoylphenoxy)ethyl]phenyl}-2-{[2-(4-hydroxyphenyl)ethyl]thio}-propanoate

Boron trifluoride etherate (128.3 mg, 0.90 mmol) was added dropwise to a solution of methyl 3-{4-[2-(4-benzoylphenoxy)ethyl]phenyl}-2-({2-(4-(benzyloxy)phenyl]ethyl}thio)propanoate (114 mg, 0.18 mmol) and dimethyl sulfide (56.2 mg, 0.90 mmol) in 4 ml dichloromethane. The reaction was stirred overnight at room temperature. Water was added and the phases were separated. The organic phase was washed (water, brine), dried (MgSO₄) and evaporated to give 78 mg of the desired product (78% yield).

¹HNMR (500 MHz, CDCl₃): 2.74-2.88 (m, 4H), 2.95 (dd, 1H), 3.13 (t, 2H), 3.22 (dd, 1H), 3.51-3.56 (m, 1H), 3.71 (s, 3H), 4.26 (t, 2H), 6.76-6.80 (m, 2H), 6.95-6.99 (m, 2H), 7.01-7.05 (m, 2H), 7.16-7.26 (m, 4H), 7.47-7.52 (m, 2H), 7.57-7.62 (m, 1H), 7.76-7.85 (m, 4H).

### f) 3-{4-[2-(4-Benzoylphenoxy)ethyl]phenyl}-2-{[2-(4-hydroxyphenyl)ethyl]thio}propanoic acid

Lithium hydroxide (6.9 mg, 0.29 mmol) dissolved in 0.5 ml water was added dropwise to an ice-cooled solution of methyl 3-{4-[2-(4-benzoylphenoxy)ethyl]phenyl}-2-{[2-(4-hydroxyphenyl)ethyl]thio}propanoate (78 mg, 0.14 mmol) in 4 ml THF. After 24 h, water was added and the solvent was evaporated. The remaining water phase was acidified with 1M HCl and extracted three times with ethyl acetate. The organic phases were pooled and washed (water, brine), dried (Na₂SO₄) and evaporated. The crude product was further purified by preparative HPLC to give 40 mg of the desired product (49% yield).

¹HNMR (400 MHz, CDCl₃): 2.72-2.98 (m, 5H), 3.10 (t, 2H), 3.18 (dd, 1H), 3.47-3.53 (m, 1H), 4.25 (t, 2H), 6.71-6.76 (m, 2H), 6.87-6.94 (m, 2H), 6.97-7.04 (m, 2H), 7.14-7.23 (m, 4H), 7.44-7.51 (m, 2H), 7.54-7.60 (m, 1H), 7.74-7.81 (m, 4H).

### Example 8

### 2-{[2-(4-Hydroxyphenyl)ethyl]thio}-3-{4-[2-(2-propylphenoxy)ethyl]phenyl}propanoic acid

### a) Methyl 2-({2-[4-(benzyloxy)phenyl]ethyl}thio)-3-{4-[2-(2-propylphenoxy)ethyl]-phenyl}propanoate

Triphenylphosphine (157 mg, 0.60 mmol) was added to a solution of 2-n-propylphenol (74 mg, 0.54 mmol) and methyl 2-({2-[4-(benzyloxy)phenyl]ethyl}thio)-3-[4-(2-hydroxyethyl)phenyl]propanoate (See Example 7 c) (245 mg, 0.54 mmol) in 6 ml toluene. The mixture was heated to 55° C. and diisopropylazodicarboxylate (121 mg, 0.60 mmol) was added. The reaction was stirred at 55° C. for 24 h. The solvent was evaporated and the crude residue was purified by preparative hplc to give 241 mg of the desired product (77% yield).

¹HNMR (400 MHz, CDCl₃): 0.96 (t, 3H), 1.52-1.61 (m, 2H), 2.57 (t, 2H), 2.76-2.90 (m, 4H), 2.95 (dd, 1H), 3.09 (t, 2H), 3.22 (dd, 1H), 3.52-3.57 (m, 1H), 3.70 (s, 3H), 4.17 (t, 2H), 5.07 (s, 2H), 6.79-6.96 (m, 4H), 7.08-7.18 (m, 6H), 7.22-7.29 (m, 2H), 7.33-7.49 (m, 5H).

### b) Methyl 2-{[2-(4-hydroxyphenyl)ethyl]thio}-3-{4-[2-(2-propylphenoxy)ethyl]phenyl}-propanoate

Boron trifluoride etherate (146 mg, 1.03 mmol) was added dropwise to a solution of methyl 2-({2-[4-(benzyloxy)phenyl]ethyl}thio)-3-{4-[2-(2-propylphenoxy)ethyl]phenyl}propanoate (117 mg, 0.21 mmol) and dimethyl sulfide (63.9 mg, 1.03 mmol) in 4 ml dichloromethane. The reaction was stirred overnight at room temperature. Water was added and the phases were separated. The organic phase was washed (water, brine), dried (MgSO₄) and evaporated. The crude product was further purified by preparative hplc to give 54 mg of the desired product (54% yield).

¹HNMR (400 MHz, CDCl₃): 0.95 (t, 3H), 1.50-1.61 (m, 2H), 2.57 (t, 2H), 2.75-2.87 (m, 4H), 2.94 (dd, 1H), 3.09 (t, 2H), 3.21 (dd, 1H) 3.50-3.57 (m, 1H), 3.69 (s, 3H), 4.17 (t, 2H), 6.73-6.77 (m, 2H), 6.79-6.91 (m, 2H), 6.99-7.05 (m, 2H), 7.11-7.18 (m, 4H), 7.21-7.26 (m, 2H).

### c) 2-{[2-(4-Hydroxyphenyl)ethyl]thio}-3-{4-[2-(2-propylphenoxy)ethyl]phenyl}propanoic acid

Lithium hydroxide (5 mg, 0.20 mmol) dissolved in 0.3 ml water was added dropwise to an ice cooled solution of methyl 2-{[2-(4-hydroxyphenyl)ethyl]thio}-3-{4-[2-(2-propylphenoxy)ethyl]phenyl}propanoate (50 mg, 0.10 mmol) in 3 ml THF. After 24 h water was added and the solvent was evaporated. The remaining water phase was acidified with 1M HCl and extracted three times with ethyl acetate. The organic phases were pooled and washed (water, brine) and dried (Na₂SO₄). The crude product was further purified by preparative HPLC to give 44 mg of the desired product (87% yield).

¹HNMR (500 MHz, CDCl₃): 0.94 (t, 3H), 1.51-1.60 (m, 2H), 2.57 (t, 2H), 2.72-2.98 (m, 5H), 3.08 (t, 2H), 3.20 (dd, 1H), 3.47-3.53 (m, 1H), 4.16 (t, 2H), 6.69-6.75 (m, 2H), 6.79-6.83 (m, 1H), 6.85-6.91 (m, 1H), 6.97-7.03 (m, 2H), 7.09-7.19 (n, 4H), 7.21-7.26 (m, 2H).

### Example 9

### 2-{[2-(4-Hydroxyphenyl)ethyl]sulfonyl}-3-[4-(2-{4-[(methylsulfonyl)oxy]phenoxy}-ethyl)phenyl]propanoic acid

### a) Methyl 2-({2-[4-(benzyloxy)phenyl]ethyl}sulfonyl)-3-[4-(2-{4-[(methylsulfonyl)oxy]phenoxy}ethyl)phenyl]propanoate

3-Chloroperbenzoic acid (198.6 mg, 0.81 mmol) was added to an ice cooled solution of methyl 2-({2-[4-(benzyloxy)phenyl]ethyl}thio)-3-[4-(2-{4-[(methylsulfonyl)oxy]phenoxy}ethyl)-phenyl]propanoate (Example 6)(200 mg, 0.32 mmol) in 24 ml dichloromethane. The reaction was stirred at room temperature for 2 hours. The mixture was washed with 1M NaHCO₃ and brine, dried (Na₂SO₄) and evaporated under reduced pressure to give 233 mg of the desired product as a yellow oil (99%).

¹HNMR (500 MHz, CDCl₃): 3.08 (t, 2H), 3.12 (s, 3H), 3.14-3.19 (m, 2H), 3.28-3.39 (m, 2H), 3.42-3.52 (m, 2H), 3.74 (s, 3H), 4.01-4.06 (m, 1H), 4.15 (t, 2H), 5.08 (s, 2H), 6.88-6.93 (m, 2H), 6.95-6.99 (m, 2H), 7.13-7.26 (m, 8H), 7.33-7.48 (m, 5H)

### b) Methyl 2-{[2-(4-hydroxyphenyl)ethyl]sulfonyl}-3-[4-(2-{4-[(methylsulfonyl)oxy]phenoxy}ethyl)phenyl]propanoate

Boron trifluoride etherate (130.5 mg, 0.92 mmol) was added dropwise during 5 min to a solution of methyl 2-({2-[4-(benzyloxy)phenyl]ethyl}sulfonyl)-3-[4-(2-{4-[(methylsulfonyl)oxy]phenoxy}ethyl)phenyl]propanoate (120 mg, 0.18 mmol) and dimethyl sulfide (57.1 mg, 0.92 mmol) in 5 ml dichloromethane. The reaction was stirred overnight at room temperature. Water was added and the phases were separated. The organic phase was washed (water, brine), dried (MgSO₄) and evaporated. The crude product was further purified by preparative hplc to give 82 mg of the desired product (71% yield).

¹HNMR (400 MHz, CDCl₃): 3.03-3.16 (m, 7H), 3.22-3.47 (m, 4H), 3.74 (s, 3H), 3.99-4.05 (m, 1H), 4.14 (t, 2H), 6.76-6.81 (m, 2H), 6.87-6.91 (m, 2H), 7.06-7.25 (m, 8H).

### c) 2-{[2-(4-Hydroxyphenyl)ethyl]sulfonyl}-3-[4-(2-{4-[(methylsulfonyl)oxy]phenoxy}ethyl)phenyl]propanoic acid

Lithium hydroxide (6.98 mg, 0.29 mmol) dissolved in 0.5 ml water was added dropwise to an ice-cooled solution of methyl 2-{[2-(4-hydroxyphenyl)ethyl]sulfonyl}-3-[4-(2-{4-[(methylsulfonyl)oxy]phenoxy}ethyl)phenyl]propanoate (82 mg, 0.145 mmol) in 5 ml THF. After 48 h water was added and the solvent was evaporated. The remaining water phase was acidified with 1M HCl and extracted three times with ethyl acetate. The organic phases were pooled and washed (water, brine) and dried (Na₂SO₄). The crude product was further purified by preparative HPLC to give 47 mg of the desired product (54% yield).

¹HNMR (400 MHz, (CD₃)₂CO): 3.02-3.08 (m, 4H), 3.21 (s, 3H), 3.34 (t, 2H), 3.41-3.60 (m, 2H), 4.21 (t, 2H), 4.23-4.27 (m, 1H), 6.77-6.81 (m, 2H), 6.98-7.01 (m, 2H), 7.12-7.16 (m, 2H), 7.23-7.28 (m, 6H).

### Example 10

### 2-{[2-(4-Hydroxyphenyl)ethyl]thio}-3-[3-(2-{4-[(methylsulfonyl)oxy]phenoxy}ethyl)-phenyl]propanoic add

### a) 2-(3-aminophenyl)ethanol

3-Nitrophenethyl alcohol (4.8 g, 28.7 mmol) was dissolved in 117 ml ethyl acetate. A small amount of 5% palladium on activated carbon was added. The reaction was stirred overnight at room temperature under hydrogen atmosphere. According to thin layer chromatography all the starting material was consumed. The mixture was filtered through Celite and the solvent was evaporated to give 3.7 g (93% yield) of the desired product.

¹HNMR (400 MHz, CDCl₃): 2.76 (t, 2H), 3.81 (t, 2H), 6.52-6.57 (m, 2H), 6.62 (d, 1H), 7.06-7.11 (m, 1H).

### b) Methyl 2-chloro-3-[3-(2-hydroxyethyl)phenyl]propanoate

2-(3-aminophenyl)ethanol (3.01 g, 21.9 mmol), acetone (53 ml) and conc. HCl (9 ml) was mixed and cooled to 0° C. Sodium nitrite (1.86 g, 21.9 mmol) dissolved in 4 ml water was added. The temperature was kept under 0° C. After 1 h methyl acrylate (18.9 g, 219.4 mmol) was added and then Cu(I)I (0.42 g, 2.19 mmol) in portions. The mixture was stirred at room temperature over night. The acetone was evaporated and water was added. The water phase was extracted three times with ethyl acetate. The organic phases were pooled and washed (water, brine), dried (MgSO₄) and evaporated. The crude product was purified by flash chromatography using a 60:40 mixture of ethyl acetate/toluene as eluent to yield 2.95 g of the desired product (yield 55%).

¹HNMR (400 MHz, CDCl₃): 2.85 (t, 2H), 3.16 (dd, 1H), 3.35 (dd, 1H), 3.74 (s, 3H), 3.85 (t, 2H), 4.45 (t, 1H), 7.05-7.28 (m, 4H).

### c) Methyl 3-(3-{2-[4-(benzyloxy)phenoxy]ethyl}phenyl)-2-chloropropanoate

To a solution of methyl 2-chloro-3-[3-(2-hydroxyethyl)phenyl]propanoate (2.95 g, 12.2 mmol) and 4-(benzyloxy)phenol (2.44 g, 12.2 mmol) in dry toluene (50 ml) was added triphenylphosphine (3.51 g, 13.4 mmol). The mixture was heated to 55° C. and diisopropylazodicarboxylate (2.70 g, 13.4 mmol) was added. The reaction was stirred over night at 55° C. under nitrogen atmosphere.

The solvent was evaporated under reduced pressure and the residual oil was purified by flash chromatography using toluene as eluent to give 2.66 g of the desired product (51% yield).

¹HNMR (400 MHz, CDCl₃): 3.05 (t, 2H) 3.15 (dd, 1H), 3.36 (dd, 1H), 3.72 (s, 3H), 4.11 (t, 2H), 4.44 (t, 1H), 5.01 (s, 2H), 6.80-6.84 (m, 2H), 6.87-6.91 (m, 2H), 7.06-7.20 (m, 4H), 7.23-7.43 (n, 5H).

### d) Methyl 2-chloro-3-{3-[2-(4-hydroxyphenoxy)ethyl]phenyl}propanoate

Methyl 3-(3-{2-[4-(benzyloxy)phenoxy]ethyl}phenyl)-2-chloropropanoate (2.66 g, 6.26 mmol) and dimethyl sulfide (1.94 g, 31.3 mmol) was dissolved in dichloromethane (80 ml). Boron trifluoride diethyl etherate (4.44 g, 31.3 mmol) was added dropwise. The reaction mixture was stirred at room temperature for 36 hours. Water was added, the phases were separated and the water phase was extracted twice with dichloromethane. The organic phases were pooled, washed with water and brine, dried (MgSO₄) and evaporated under reduced pressure to give 2.03 g (96% yield) of the desired product.

¹HNMR (400 MHz, CDCl₃): 3.04 (t, 2H), 3.15 (dd, 1H), 3.35 (dd, 1H), 3.73 (s, 3H), 4.10 (t, 2H), 4.45 (t, 1H), 6.72-6.79 (m, 4H), 7.07-7.27 (m, 4H).

### e) Methyl 2-chloro-3-[3-(2-{4-[(methylsulfonyl)oxy]phenoxy}ethyl)phenyl]propanoate

To a solution of methyl 2-chloro-3-{3-[2-(4-hydroxyphenoxy)ethyl]phenyl}propanoate (2.03 g, 6.06 mmol) in dichloromethane was added triethylamine (1.84 g, 18.2 mmol). The mixture was cooled to −20° C. under nitrogen atmosphere. Methanesulfonyl chloride (0.69 g, 6.06 mmol) was added dropwise. The mixture was stirred until it reached room temperature. Dichloromethane was added. The organic phase was washed twice with water, dried (MgSO₄) and evaporated. The crude product was purified by flash chromatography using a 99.5:0.5 mixture of dichloromethane/methanol as eluent to give 2.02 g of the desired product (80% yield).

¹HNMR (400 MHz, CDCl₃): 3.07 (t, 2H), 3.10 (s, 3H), 3.16 (dd, 1H), 3.36 (dd, 1H), 3.73 (s, 3H), 4.15 (t, 2H), 4.44 (t, 1H), 6.86-6.91 (m, 2H), 7.08-7.14 (m, 2H), 7.15-7.20 (m, 2H), 7.24-7.28 (m, 2H).

### f) Methyl 2-({2-[4-(benzyloxy)phenyl]ethyl}thio)-3-[3-(2-{4-[(methylsulfonyl)oxy]phenoxy}ethyl)phenyl]propanoate

S-{2-[4-(benzyloxy)phenyl]ethyl}ethanethioate (1.40 g, 4.89 mmol) and methanol (9 ml) was added to a 2-necked round bottomed flask flushed with argon. To this slurry was added sodiumthiomethoxide (0.34 g, 4.89 mmol) dissolved in 5 ml methanol. The mixture was stirred at room temperature for ˜30 min. Half the solvent volume was evaporated under reduced pressure. To the concentrated reaction mixture was added methyl 2-chloro-3-[3-(2-{4-[(methylsulfonyl)oxy]phenoxy}ethyl)phenyl]propanoate (2.02 g, 4.89 mmol) dissolved in 4.5 ml dry DMF. The mixture was stirred at room temperature for ˜30 min under an argon atmosphere. The solvent was evaporated and the residue was dissolved in ethyl acetate. The organic phase was extracted five times with water and once with brine, dried (MgSO₄) and evaporated. The crude product (3 g) was further purified by preparative HPLC using a gradient of CH₃CN/5% CH3CN-waterphase containing 0.1M NH₄OAc, 40% CH₃CN=>100% CH₃CN in 50 min, to give 2.56 g (84% yield) of the desired product.

¹HNMR (400 MHz, CDCl₃): 2.74-2.85 (m, 4H), 2.92 (dd, 1H), 3.05 (t, 2H), 3.08 (s, 3H), 3.18 (dd, 1H), 3.47-3.53 (m, 1H), 3.65 (s, 3H), 4.12 (t, 2H), 5.03 (s, 2H), 6.85-6.91 (m, 4H), 7.04-7.43 (m, 13H).

### g) Methyl 2-{[2-(4-hydroxyphenyl)ethyl]thio}-3-[3-(2-{4-[(methylsulfonyl)oxy]phenoxy}-ethyl)phenyl]propanoate

Methyl 2-({2-[4-(benzyloxy)phenyl]ethyl}thio)-3-[3-(2-{4-[(methylsulfonyl)oxy]phenoxy}-ethyl)phenyl]propanoate (2.56 g, 4.12 mmol) and dimethyl sulfide (1.28 g, 20.6 mmol) was dissolved in dichloromethane (80 ml). Boron trifluoride diethyl etherate (2.92 g, 20.6 mmol) was added drop wise. The reaction was stirred for 2 days at room temperature under nitrogen atmosphere. Water was added and the phases were separated. The water phase was extracted twice with dichloromethane. The organic phases were pooled and washed once with water, dried (MgSO₄) and evaporated to give 1.99 g (91% yield) of the desired product.

¹HNMR (400 MHz, CDCl₃): 2.70-2.84 (m, 4H), 2.92 (dd, 1H), 3.05 (t, 2H), 3.10 (s, 3H), 3.18 (dd, 1H), 3.45-3.50 (m, 1H), 3.66 (s, 3H), 4.12 (t, 2H), 6.70-6.73 (m, 2H), 6.85-6.89 (m, 2H), 6.97-7.00 (m, 2H), 7.03-7.10 (m, 2H), 7.12-7.25 (m, 4H).

### h) 2-{[2-(hydroxyphenyl)ethyl]thio}-3-[3-(2-{4-[(methylsulfonyl)oxy]phenoxy}ethyl)-phenyl]propanoic acid

Methyl 2-{[2-(4-hydroxyphenyl)ethyl]thio}-3-[3-(2-{4-[(methylsulfonyl)oxy]phenoxy}-ethyl)phenyl]propanoate (1.99 g, 3.75 mmol) was dissolved in THF (13.1 ml) and cooled in an ice-bath. Lithium hydroxide (0.45 g, 18.75 mmol) dissolved in 1.9 ml water was added dropwise. The reaction was stirred at room temperature overnight. Water was added and the THF was evaporated. The remaining water phase was acidified with 1M HCl and extracted with ethyl acetate three times. The organic phases were pooled and washed with brine, dried (MgSO₄) and evaporated. The crude product was further purified by preparative HPLC using a gradient of CH₃CN/5% CH₃CN-waterphase containing 0.1M NH₄OAc, 20% CH₃CN=>100% CH₃CN in 40 minutes, to give 1.4 g of the desired product (76% yield).

¹HNMR (400 MHz, CD₃OD): 2.67-2.88 (m, 5H), 3.03 (t, 2H), 3.11 (dd, 1H), 3.13 (s, 3H), 3.42-3.47 (m, 1H), 4.16 (t, 2H), 6.65-6.68 (m, 2H), 6.90-6.97 (m, 4H), 7.05-7.09 (m, 1H), 7.12-7.22 (m, 5H).

### Example 11

### 3-{4-[2-(2-benzyl-4-methanesulfonyloxyphenoxy)ethyl]phenyl}-2-[2-(4-hydroxyphenyl)ethylsulfanyl]-propionic acid

Lithium hydroxide (62 mg, 2.58 mmol) in water (7.5 ml) was added to a stirred solution of 3-{4-[2-(2-benzyl-4-methanesulfonyloxyphenoxy)ethyl]phenyl}-2-[2-(4-hydroxy-phenyl)-ethylsulfanyl]-propionic acid methyl ester (234 mg, 0.377 mmol prepared by analogous methods to the previous examples) in THF (15 ml) at room temperature. After 16 h 2M Potassium hydrogensulfate solution (5 ml, 10 mmol) was added and the THF was removed under reduced pressure. Dilution with water and extraction with methylene chloride, drying of the organic phase (magnesium sulfate) and evaporation of the solvent gave a residue which was purified by preparative HPLC (acetonitrile/ammonium acetate buffer) to give 3-{4-[2-(2-benzyl-4-methanesulfonyloxyphenoxy)ethyl]phenyl}-2-[2-(4-hydroxyphenyl)ethylsulfanyl]-propionic acid. Yield 66 mg (29%).

NMR (CDCl3, 500 MHz) δ 7.31-7.08 (m, 9), 7.06-7.03 (m, 1), 6.95 (d, 1), 6.83 (d, 2), 6.71 (d, 1), 6.65 (d, 2), 4.04 (t, 2), 3.89 (s, 2), 3.50 (“t”, 1), 3.19-3.10 (m, 1), 3.01 (s, 3), 2.99-2.94 (t, 2), 2.91-2.83 (m, 1), 2.80-2.58 (m, 4).

### Example 12

### 2-[2-(4-tert-Butoxy-phenyl)-ethylsulfanyl]-3-{4-[2-(4-methanesulfonyloxyphenoxy)ethyl]-phenyl}propionic acid

2-[2-(4-tert-Butoxy-phenyl)-ethylsulfanyl]-3-{4-[2-(4-methanesulfonyloxyphenoxy)ethyl]-phenyl}propionic acid methyl ester (51 mg, 0.087 mmol, prepared by analogous methods to the previous examples) was stirred overnight with lithium hydroxide (3 mg, 0.13 mmol) in THF (0.5 ml) and water (0.1 ml). Water was added and THF was removed in vacuo. The mixture was washed with diethyl ether and the aqueous phase was acidified with potassium hydrogensulfate and then extracted with methylene chloride. Preparative HPLC (acetonitrile/ammonium acetate buffer) yielded 2-[2-(4-tert-butoxy-phenyl)-ethylsulfanyl]-3-{4-[2-(4-methanesulfonyloxyphenoxy)ethyl]-phenyl}propionic acid (16 mg, 32%). NMR (CDCl3, 500 MHz) δ 7.20-7.15 (m, 6), 7.04 (d, 2), 6.88 (dd, 4), 4.13 (t, 2), 3.51 (t, 1), 3.22-3.14 (m, 1), 3.10 (s, 3), 3.05 (t, 2), 2.96-2.73 (m, 5), 1.33 (s, 9).

Biological Activity

The compounds of the invention were tested in the assays described in WO03/051821 which is incorporated herein by reference. For example the EC₅₀ of Example 7 for human PPAR alpha is 0.78 μmol/l.

The compounds of formula I have an affinity for PPARα and/or PPARγ. The compounds of formula I are selected because of their superior potency in vitro and/or higher affinity and/or higher in vivo efficacy. The compounds also have a better selectivity profile, which is expected to improve in vivo safety.

In addition the compounds of the present invention may have improved DMPK (Drug Metabolism and Pharmacokinetic) properties, for example improved metabolic stability in vitro or bioavailability. The compounds also have an improved solubility and/or a promising toxicological profile.

1. A compound of formula I 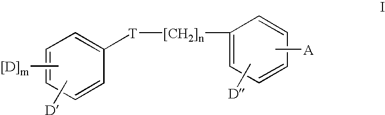
and pharmaceutically acceptable salts thereof, in which
A is situated in the para position and represents A1 or A2 below 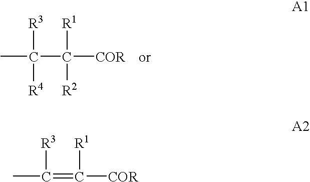
wherein
R is hydrogen;
—OR^{a}, wherein R^{a} represents hydrogen, alkyl, aryl or alkylaryl;
—NR^{a}R^{b}, wherein R^{a} and R^{b} are the same or different and R^{a} is as defined above and R^{b} represents hydrogen, alkyl, aryl, alkylaryl, cyano, —OH, -Oalkyl, -Oaryl, -Oalkylaryl, —COR^{c} or —SO₂R^{d}, wherein R^{c} represents hydrogen, alkyl, aryl or alkylaryl and R^{d} represents alkyl, aryl or alkylaryl;
R¹ is alkyl, aryl, alkenyl, alkynyl, cyano;
—OR^{e}, wherein R^{e} is alkyl, acyl aryl or alkylaryl;
—O—[CH₂]ₘ—OR^{f}, wherein R^{f} represents hydrogen, alkyl, acyl, aryl or alkylaryl and m represents an integer 1-8;
—OCONR^{a}R^{c}, wherein R^{a} and R^{c} are as defined above;
—SR^{d}, wherein R^{d} is as defined above;
—SOR^{d}, wherein R^{d} is as defined above;
—SO₂R^{d}, wherein R^{d} is as defined above;
—SO₂NR^{a}R^{f}, wherein R^{f} and R^{a} are as defined above;
—SO₂OR^{a}, wherein R^{a} is as defined above;
—COOR^{d}, wherein R^{d} is as defined above;
R² is hydrogen, alkyl, aryl, or alkylaryl,
R³ and R⁴ are the same or different and each represents hydrogen, alkyl, aryl, or alkylaryl,
n is an integer 1-6,
m is an integer 0 or 1;
D is situated in the ortho, meta or para position and represents alkyl, acyl, aryl, alkylaryl, halogen, —CN and NO₂, wherein the alkyl, aryl, or alkylaryl group is optionally substituted by R^{b};
—NR^{c}COOR^{a}, wherein R^{c} and R^{a} are as defined above;
—NR^{c}COR^{a}, wherein R^{c} and R^{a} are as defined above;
—NR^{c}R^{a}, wherein R^{c} and R^{a} are as defined above;
—NR^{c}SO₂R^{d}, wherein R^{c} and R^{d} are as defined above;
—NR^{c}CONR^{k}R^{c}, wherein R^{a}, R^{c} and R^{k} are as defined above;
—NR^{c}CSNR^{a}R^{k}, wherein R^{a}, R^{c} and R^{k} are as defined above;
—ORₐ, wherein R^{a} is as defined above;
—OSO₂R^{d}, wherein R^{d} is as defined above;
—SO₂R^{d}, wherein R^{d} is as defied above;
SOR^{d}, wherein R^{d} is as defined above;
—SR^{c}, wherein R^{c} is as defined above;
—SO₂NR^{a}R^{f}, wherein R^{f} and R^{a} are as defined above;
—SO₂OR^{a}, wherein R^{a} is as defined above;
—CONR^{c}R^{a}, wherein R^{c} and R^{a} are as defined above;
—OCONR^{f}R^{a}, wherein R^{f} and R^{a} are as defined above;
D′ is situated in the ortho, meta or para position and represents hydrogen, alkyl, acyl, aryl, alkylaryl, halogen, —CN, —NO₂,
—NR^{f}R^{b}, wherein R^{f} and R^{b} are as defined above;
—OR^{f}, wherein R^{f} is as defined above;
—OSO₂R^{d}, wherein R^{d} is as defined above;
D″ is situated in the ortho, meta or para position and represents
hydrogen, alkyl, acyl, aryl, alkylaryl, halogen, —CN, —NO₂, —NR^{f}R^{b} wherein R^{f} and R^{b} are as defined above;
—OR^{f}, wherein R^{f} is as defined above.
—OSO₂R^{d}, wherein R^{d} is as defined above
and T represents O, S or NR^{t} wherein R^{t} represents alkyl or alkylaryl provided that when A is A1 and R², R³, and R⁴ each represent hydrogen and R¹ is OR^{e} wherein R^{e} is as previously defined then T is not O;
wherein the term “aryl” denotes a substituted or unsubstituted phenyl, furyl, thienyl or pyridyl group, or a fused ring system of any of these groups;
wherein the term “alkyl” denotes a straight or branched, substituted or unsubstituted alkyl group having from 1 to 6 carbon atoms or a substituted or unsubstituted cycloalkyl having from 3 to 6 carbon atoms and wherein the term “substituted” denotes substitution by one or more alkyl, alkoxy, halogen, thiol, nitro, hydroxy, acyl, aryl or cyano groups or an amino group optionally substituted by one or two alkyl groups:
with a first proviso that when D is CH₃S(O)₂O and m is 1 and D′ is H and T is O and n=2 and A is a group CH₂CH(SCH₂CH₂Ph)COR^{x} in which the phenyl is substituted in the 4 position by OH, Cl or F and in which R^{x} represents OH, or a protecting group for a carboxylic hydroxy group including a ethoxy or benzyloxy then D″ is not H;
and with a second proviso that when m is 1 and D is CH₃S(O)₂O and D′ is H and T is O, S or NR and wherein R represents a H, a C₁₋₆alkyl group or a phenyl C₁₋₆alkyl group and n=2 and A is a group CH₂CH(OC₂H₅)COR^{x} in which R^{x} represents OH, or a protecting group for a carboxylic hydroxy group including a C₁₋₆alkoxy group or benzyloxy then D″ is not H.

2. A compound of formula I as claimed in claim 1 in which A represents a group of formula —CH₂—CH(CO₂H)—S(O)ₚ—(CH₂)_{q}—Ar wherein p is 0, 1 or 2; q is 1, 2, 3 or 4; and Ar is phenyl or thienyl each of which is optionally substituted by one or more hydroxy, C₁₋₆alkyl, C₁₋₆alkoxy, halogen, cyano or an amino group optionally substituted by one or two alkyl groups.

3. A compound of formula I as claimed in claim 1 represented by formula IA 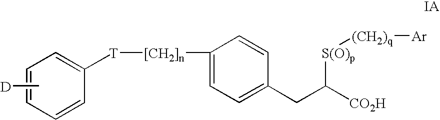
or a pharmaceutically acceptable salt thereof in which
D represents C₁₋₆alkylsulfonyloxy, aroyl, or a C₁₋₆alkyl group;
T represents O, S or NR^{t} wherein R^{t} represents alkyl or alkylaryl;
n is 1, 2 or 3;
p is 0, 1 or 2;
q is 1 or 2; and
Ar is phenyl or thienyl each of which is optionally substituted by hydroxy, C₁₋₆alkyl, C₁₋₆alkoxy, halogen, cyano or an amino group optionally substituted by one or two alkyl groups and wherein the group containing the carboxylic acid group is attached to the phenyl ring meta or para to the group (CH₂)n-T-.

4. A compound selected from one or more of the following:
2-[(4-cyanobenzyl)thio]-3-[4-(2-{4-[(methylsulfonyl)oxy]phenoxy}ethyl)-phenyl]propanoic acid;
2-({2-[4-(dimethylamino)phenyl]ethyl}thio)-3-[4-(2-{4-[(methylsulfonyl)oxy]phenoxy}-ethyl)phenyl]propanoic acid;
3-[4-(2-{4-[(methylsulfonyl)oxy]phenoxy}ethyl)phenyl]-2-{[2-(2-thienyl)ethyl]thio}-propanoic acid;
2-{[2-(2-fluorophenyl)ethyl]thio}-3-[4-(2-{4-[(methylsulfonyl)oxy]phenoxy}-ethyl)phenyl]-propanoic acid;
2-{[2-(3-methoxyphenyl)ethyl]thio}-3-[4-(2-{4-[(methylsulfonyl)oxy]phenoxy}-ethyl)phenyl]propanoic acid;
2-{[2-(4-hydroxyphenyl)ethyl]sulfinyl}-3-[4-(2-{4-[(methylsulfonyl)oxy]phenoxy}-ethyl)phenyl]propanoic acid;
3-{4-[2-(4-benzoylphenoxy)ethyl]phenyl}-2-{[2-(4-hydroxyphenyl)ethyl]thio}propanoic acid;
methyl 2-({2-[4-(benzyloxy)phenyl]ethyl}thio)-3-{4-[2-(2-propylphenoxy)ethyl]phenyl}-propanoate;
2-{[2-(4-hydroxyphenyl)ethyl]thio}-3-{4-[2-(2-propylphenoxy)ethyl]phenyl}propanoic acid;
2-{[2-(4-hydroxyphenyl)ethyl]thio}-3-[3-(2-{4-[(methylsulfonyl)oxy]phenoxy}-ethyl)phenyl]propanoic acid;
3-{4-[2-(2-benzyl-4-methanesulfonyloxyphenoxy)ethyl]phenyl}-2-[2-(4-hydroxyphenyl)-ethylsulfanyl]propionic acid; and
2-[2-(4-tert-butoxy-phenyl)ethylsulfanyl]-3-{4-[2-(4-methanesulfonyloxyphenoxy)ethyl]-phenyl}propionic acid
and pharmaceutically acceptable salts thereof.

5. A pharmaceutical formulation comprising a compound according to any one of claims 1 to 4 in admixture with pharmaceutically acceptable adjuvants, diluents and/or carriers.

6. A pharmaceutical formulation comprising a compound according to any one of claims 1 to 4 in admixture with pharmaceutically acceptable adjuvants, diluents and/or carriers.

7. A method of treating or preventing lipid disorders (dyslipidemia) whether or not associated with insulin resistance comprising the administration of a compound according to any one of claims 1 to 4 to a mammal in need thereof.

8. The use of a compound according to any one of claims 1 to 4 in the manufacture of a medicament for the treatment of lipid disorders (dyslipidemia) whether or not associated with insulin resistance.

9. A method of treating or preventing type 2 diabetes comprising the administration of an effective amount of a compound of formula I according to any one of claims 1 to 4 to a mammal in need thereof.

10. A pharmaceutical composition comprising a compound as claimed in any one of claims 1 to 4 combined with another therapeutic agent that is useful in the treatment of disorders associated with the development and progress of atherosclerosis such as hypertension, hyperlipidaemias, dyslipidaemias, diabetes and obesity.

11. A process for preparing a compound of formula I as claimed in claim 1 by reacting a compound of formula II 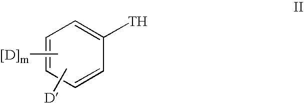
in which D, m, D′ and T are as previously defined with a compound of formula III 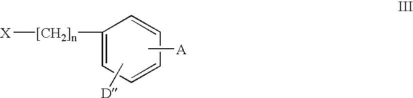
in which n, A and D″ are as previously defined and X is a leaving group for example halo or methanesulphonyloxy at a temperature in the range of 0-150° C. optionally in the presence of an inert sovent.

12. A process to prepare a compounds of formula IA as claimed in claim 3 by reacting a compound of formula IB 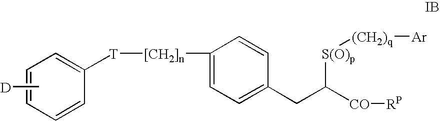
in which D, T, n, p, q and Ar are as previously defined and R^{p} represents a protecting group for a carboxylic hydroxy group with a de-protecting agent.
